SEQUENCE LISTING (6 sequences)

SEQ ID NO: 1 - protein, 9 aa, Homo sapiens
PRSFFRLGK

SEQ ID NO: 2 - protein, 8 aa, Homo sapiens
KPACFFRL

SEQ ID NO: 3 - protein, 8 aa, Homo sapiens
DPAKFFRL

SEQ ID NO: 4 - protein, 8 aa, Homo sapiens
KPMKFFRL

SEQ ID NO: 5 - protein, 8 aa, Homo sapiens
LPAKFFRL

SEQ ID NO: 6 - protein, 7 aa, Homo sapiens
AIPVSLR

(19) United States
(12) Patent Application Publication
(10) Pub. No.: US 2023/0321265 A1
(43) Pub. Date: Oct. 12, 2023

(54) KAPPA OPIOID RECEPTOR AGONIST-THERAPEUTIC ANTIGEN BINDING PROTEIN CONJUGATE (KTAC) COMPOUNDS

(71) Applicant: Cara Therapeutics, Inc., Stamford, CT (US)

(72) Inventors: Derek T CHALMERS, Riverside, CT (US); Michael E. LEWIS, Kennett Square, PA (US)

(21) Appl. No.: 18/007,701

(22) PCT Filed: Jun. 1, 2021
(86) PCT No.: PCT/US2021/035084

§ 371 Date: Dec. 1, 2022

Related U.S. Application Data
(60) Provisional application No. 63/031,843, filed on May 29, 2020.

(51) Int. Cl. A61K 47/68; A61P 29/00

(52) U.S. Cl. CPC A61K 47/6811; A61P 29/00

(57) ABSTRACT

Conjugate (KTAC) compounds that include a peptidic kappa opioid receptor agonist linked through a protease-sensitive linker and/or acid-sensitive linker to an antibody molecule are provided. The linker may include spacer regions on either side of the protease sensitive cleavage site. The antibody can be a monoclonal antibody such as a therapeutic monoclonal antibody. The KTAC compounds may be incorporated into pharmaceutical compositions useful for the treatment of diseases and conditions, particularly those that include an inflammatory component.

### FIELD OF THE INVENTION

The invention relates to KTAC compounds that include a kappa opioid receptor agonist covalently bound to a therapeutic antigen binding protein, such as a therapeutic antibody or a therapeutic antigen receptor fragment, through a linker containing a protease cleavage site or an acid-labile linkage. The invention also relates to the use of peripherally-restricted selective peptide kappa opioid agonists conjugated to therapeutic antigen binding proteins to treat inflammation associated with various disease states.

### RELATED ART

The kappa opioid receptor is one of a family of seven transmembrane-spanning G protein-coupled receptors (mu, kappa and delta opioid receptors) that are activated by binding endogenously produced opioid peptides as well as exogenously administered opioid compounds. Kappa opioid receptor agonists preferentially bind to the kappa opioid receptor embedded in cellular membranes and initiate intracellular signal transduction events leading to a range of effects, including analgesia and a reduction in inflammation and pruritus. Kappa opioid receptors are found in the central nervous system, on peripheral sensory neurons, and on cells of the immune system, as well as several other specific types of non-neural cells. For example, kappa opioid receptors are present in human synovial tissue, notably in fibroblast-like synoviocytes, where they are down-regulated in patients with osteoarthritis and rheumatoid arthritis, but can be up-regulated in response to a non-peptide kappa opioid agonist (Shen et al. Arthritis & Rheumatism, vol. 52, May 2005, pp. 1402-1410). Kappa opioid receptors are also expressed by skin keratinocytes (See for example, Tomigawa et al. Possible roles of epidermal opioid systems in pruritus of atopic dermatitis, J. Invest. Dermatol. vol. 127, 2228-2235). Nonpeptide kappa opioid agonists can reduce inflammation and joint destruction in animal models of arthritis (Wilson et al. 2008, J. Pain 8(12) 924-930; Binder and Walker 1998 Brit. J. Pharmacol. Vol. 124 647-654).

A novel class of D-amino acid peptide amide selective kappa opioid receptor agonists with analgesic, anti-inflammatory and anti-pruritic activity has been shown to be peripherally restricted when administered intravenously or orally due to their exclusion from the central nervous system by their inability to cross the blood-brain barrier: See U.S. Pat. Nos. 5,965,701 of Junien et al., 7,713,937 of Schteingart et al., and 10,550,150 of Desai et al., the disclosures of each of which are incorporated by reference herein in their entireties. This pharmacological selectivity and peripheral restriction leads to the distinguishing feature of these molecules as agonists of the kappa opioid receptor providing analgesic, anti-pruritic and anti-inflammatory activity while lacking the respiratory depressive, dysphoric, and addictive properties of many other opioids.

Monoclonal antibodies (Mabs) are a major class of therapeutic antigen binding proteins of 150 K-170 K molecular weight with binding sites that specifically bind a target antigen. Mabs have been used as therapeutic drugs for the treatment of a growing number of diseases and conditions. These therapeutic Mabs include, for instance, the anti-TNF Mabs, adulimumab and certolizumab, and the anti-IL13 Mab, abrezekimab, among many others well known in the art.

A variety of macromolecules and polymers, such as polyethylene glycol (PEG), have been used as moieties for the attachment of various bioactive substances. In some cases, these macromolecules are covalently attached to the bioactive substance for the purpose of prolonging its presence in the systemic circulation, without any attempt at targeting particular tissues or enabling release of the bioactive substance from its macromolecular carrier. In other cases, when tissue targeting is an objective, antibodies to antigens enriched in particular tissues, e.g., cancer cells, are coupled to bioactive substances, e.g., peptide or non-peptide toxins, that are being targeted to these tissues (see for instance, Doronina, S.O. et al., Enhanced Activity of Monomethylauristatin F through Monoclonal Antibody Delivery: Effects of Linker Technology on Efficacy and Toxicity. Bioconjugate Chem. 2006, 17, 1, 114-124). In such cases, provision is often made for release of the bioactive substance from the carrier in the microenvironment of the target tissue, e.g., via coupling the bioactive substance to the carrier with a moiety that can be cleaved by the target tissue; examples include peptide linkers that can be cleaved by tumor-specific proteases, or cleaved through a pH-sensitive cleavage reaction inside the target cells, or by a protease that is co-localized to the target cells, or alternatively cleaved by a complement-dependent cleavage reaction. See, for instance, U.S. Pat. No. 10,441,649.

Another type of antibody conjugate targets a hormone receptor or a cytokine receptor to block the activity of the cognate hormone or cytokine. For example, see U.S. Pat. No. 10,509,035 of Dubreuil et al.

Conjugation of enzymes and other molecules with antibodies, often for amplification and assay purposes, is well known in the art. See, for example, the treatise, Hermanson, G.T. (2008) Bioconjugate Techniques. Academic Press and Elsevier, 2^{nd} ed. ISBN 978-0-12-370501-3.

### SUMMARY OF THE INVENTION

The invention provides a kappa opioid receptor agonist-therapeutic antigen binding protein conjugate (KTAC) compound having the structure of formula I:

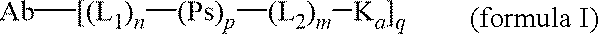

In formula I, Ab is an antigen binding protein, such as an antibody, or a fragment thereof, that has a binding site for an antigen on or in a target tissue and/or target cell or expressed in a disease or condition. The moiety Kₐ includes a kappa opioid receptor peptide agonist. The antigen binding protein or antigen binding protein fragment, Ab, is covalently bound to the moiety Kₐ, that includes the kappa opioid receptor peptide agonist through the linker (L₁)ₙ—(Ps)ₚ—(L₂)ₘ.

The operators n and m and p are each independently zero or 1; q is independently an integer from 1 to about 10. In the event that p is zero, then m is zero and (L₁)ₙ includes an acid-labile moiety (ALM). Further, when n and m are each zero, then p is equal to 1, i.e., the protease-cleavable peptide linker (Ps) must be present. The protease capable of cleaving the protease cleavable site, (Ps), can be any suitable protease, such as a tissue specific protease, such as a neutral protease, a serine protease, or a matrix metalloprotease. Alternatively, the protease capable of cleaving the protease cleavable site, (Ps), can be bacillolysin, dispase, mast cell serine protease, chymase, chymotrypsin, trypsin, tryptase, subtilisin, signal peptidase, matrix metalloproteinase 1, matrix metalloproteinase 2, matrix metalloproteinase 3 or matrix metalloproteinase 7. Each of the linkers L₁, Ps and L₂, when present, can include spacer amino acid sequences providing distance between the Ab component and the Kₐ kappa opioid receptor agonist moiety. For example, the spacer sequence or sequences can include glycine and/or alanine residues, or other amino acids that provide linear extension rather than secondary structure.

The antigen binding protein, Ab, can be a therapeutic antibody/therapeutic antibody fragment having a binding site for a tissue specific antigen or a binding site for an antigen overexpressed in a disease or condition. The disease or condition can be any disease or condition treatable with a kappa opioid receptor agonist; by way of nonlimiting examples, such as, for instance, pruritus, pain, or migraine, or any disease or condition having an inflammatory component.

The KTAC compound having the structure: Ab—[(L₁)ₙ—(Ps)ₚ—(L₂)ₘ—Kₐ]_{q} of formula I can include linker components (L₁)ₙ and (L₂)ₘ, which when present, are covalently bound to Ps, a peptide that includes at least one protease cleavage site. The linker —(L₁)ₙ—(Ps)ₚ—(L₂)ₘ— is covalently bound to the antigen binding protein or antigen binding protein fragment, Ab at one end through the linker component, —(L₁)ₙ—, and is also covalently bound to the moiety, —Kₐ which includes a kappa opioid receptor peptide agonist at the other end through the linker component, —(L₂)ₘ—.

The invention further provides a pharmaceutical composition including a KTAC compound having the structure of formula I and a pharmaceutically acceptable excipient; wherein formula I is Ab—[(L₁)ₙ—(Ps)ₚ—(L₂)ₘ—Kₐ]_{q}; and Ab is an antigen binding protein or an antigen binding protein fragment that has a binding site for an antigen in a tissue or cell present in a disease or condition. The moiety Kₐ includes a kappa opioid receptor peptide agonist. The antigen binding protein or antigen binding protein fragment, Ab, is covalently bound to the moiety Kₐ, including the kappa opioid receptor peptide agonist, through the linker —(L₁)ₙ—(Ps)ₚ—(L₂)ₘ—. See FIGS. 1, 2 and 3.

The KTAC compounds having the structure of formula I of the invention are also useful for the treatment of patients suffering from kappa opioid receptor-associated diseases and conditions such as pain, inflammation, and pruritus.

### BRIEF DESCRIPTION OF THE FIGURES

FIG. 1: Schematic of a KTAC compound that includes an IgG molecule having linkers —(L₁)—(Ps)—(L₂)— each attaching a kappa opioid receptor agonist, Kₐ. The IgG molecule has two polypeptide light chains and two polypeptide heavy chains. The light chains each consist of a variable region V_{L} contributing to the antigen binding region and a constant region C_{L} of the kappa or lambda type. The two heavy chains each consist of a variable region V_{H} contributing to the antigen binding region and a constant region divided into three sections, C_{H}1, C_{H}2 and C_{H}3. The variable and constant regions of the light and heavy chains each have intra-chain disulfide bonds (not shown). The C_{H}2 regions may be glycosylated, shown as branched chains of gray ovals representing the saccharide monomers of the polysaccharide. The VL and VH regions contribute to the antigen binding region or complementarity determining region (CDR). The kappa opioid receptor agonist Kₐ moieties are shown randomly linked to constant and variable regions of the light and heavy chains.

FIG. 2: Schematic of KTAC components including linkers —(L₁)—(Ps)—(L₂)— attached to a kappa opioid receptor agonist, Kₐ. Examples of linker components L1, Ps and L2 are shown: spacer peptides are represented as a solid line, acid labile moieties (ALMs) are represented as squares, different protease sensitive cleavage sites are represented as triangles, diamonds and ovals. The exemplified kappa opioid receptor agonist Kₐ shown is CR845. Each of L1, Ps and L2 can be present or absent. L1 and L2 can include one or more ALMs, or only spacer sequences. Ps can include single or multiple copies of one or more different protease sensitive cleavage sites.

FIG. 3: Schematic of KTAC components including multiple linkers —(L₁)—(Ps)—(L₂)—with several kappa opioid receptor agonists Kₐ attached. Examples of catenated linker components L1, Ps and L2 with two and four Ka moieties attached are shown. The Ka moieties are covalently bonded through the N-termini to side amino acid side chains of the linker peptides. L1 and L2 can include one or more ALMs, or only spacer sequences. Ps can include single or multiple copies of one or more different protease sensitive cleavage sites.

FIG. 4: Schematic of examples of different antigen-binding proteins incorporated as the Ab component of the KTACs of the invention: (a) The fully humanized IgG1 of adalimumab (Humira®); (b) The IgG of infliximab (Remicade®) having mouse heavy and light chain variable regions (shown as dark shaded regions); (c) The Fc fusion protein with the extracellular portion of the human TNF-alpha receptor (shown as circles) of etanercept (Enbrel®); and (d) the murine CDR (shown as shaded sections) within the Fab′ fragment linked to two polyethylene glycol molecules (shown as ovals) of Certulizumab (Cimzia® pergol).

FIG. 5 Reduction in LPS-induced TNF release in Mice Pretreated with SC CR845 at 1, 3 and 10 mg/kg; and separately showing a comparison of the effect of prednisone as compared with vehicle alone.

FIG. 6: Intravenous CR845 Produces Dose-Dependent Inhibition of Carrageenan-Induced Paw Edema Formation in Rats: Chart showing paw volume after administration of vehicle alone, or vehicle plus 0.1, 0.3 or 1.0 mg/kg CR845.

### DETAILED DESCRIPTION

The invention provides a kappa opioid receptor agonist-therapeutic antigen binding protein conjugate (KTAC) compound having the structure of formula I:

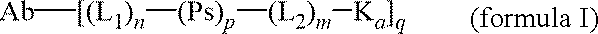

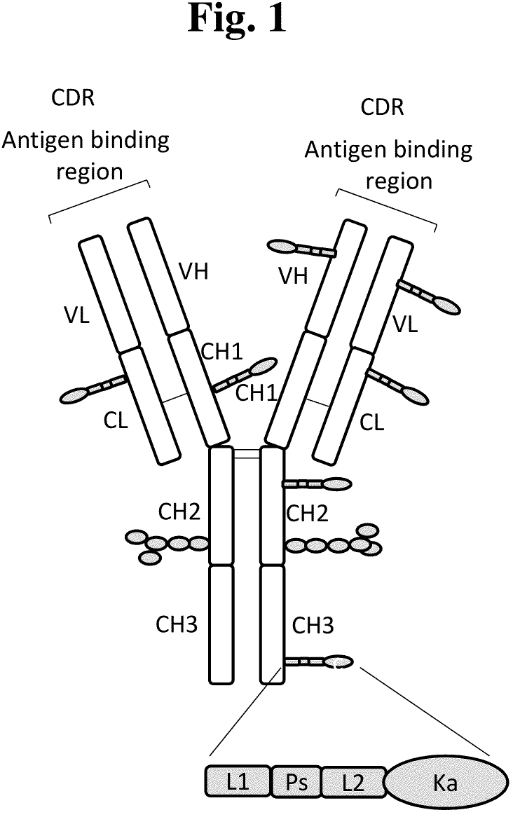

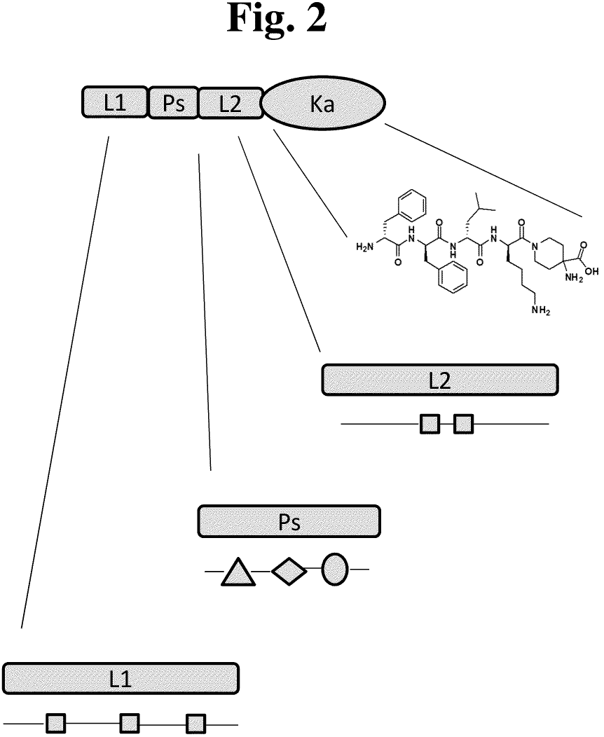

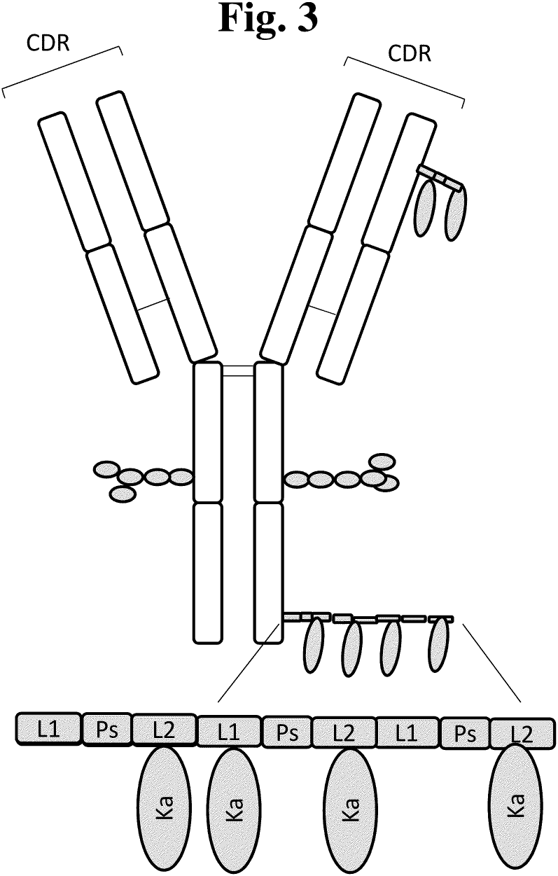

In this embodiment, formula I, Ab is an antigen binding protein, such as an antibody, or an antibody fragment, that has a binding site for an antigen, the target antigen that is relatively enriched in a tissue and/or cell. Alternatively, the target antigen may be expressed or overexpressed in a tissue and/or cell in a disease or condition to be treated by the KTAC of the invention. The moiety Kₐ includes a kappa opioid receptor peptide agonist. The antigen binding protein or fragment thereof, Ab, is covalently bound to the moiety Kₐ, including the kappa opioid receptor peptide agonist through the linker (L₁)ₙ—(Ps)ₚ—(L₂)ₘ, see FIGS. 1, 2 and 3 for examples of these structures.

The operators n and m and p are each independently zero or 1; q is independently an integer from 1 to about 10. In the event that p is zero, then m is zero and (L₁)ₙ includes an acid-labile moiety (ALM). Further, when n and m are each zero, then p is equal to 1, i.e., the protease-cleavable peptide linker (Ps) must be present.

The KTAC compound having the structure: Ab—[(L₁)ₙ—(Ps)ₚ—(L₂)ₘ—Kₐ]_{q} of formula I can include linker components (L₁)ₙ and (L₂)ₘ, which when present, are covalently bound to Ps, a peptide that includes at least one protease cleavage site. The linker —(L₁)ₙ—(Ps)ₚ—(L₂)ₘ— is covalently bound to the antigen binding protein or fragment, Ab by —(L₁)ₙ, at one end of the linker, and is also covalently bound to the moiety, Kₐ which includes a kappa opioid receptor peptide agonist, (L₂)ₘ- at the other end of the linker. When L₁ is absent, the antigen binding protein or fragment is directly covalently bound to —(Ps) or to —(L₂).

In one embodiment, the invention provides a KTAC compound having the structure of formula I wherein the Ab moiety is an antibody fragment, such as an F(ab) fragment, or an F(ab′)₂ fragment, or a single chain antibody.

In another embodiment, the invention provides a KTAC compound having the structure of formula I wherein the Ab moiety is a receptor or receptor fragment capable of binding the target antigen.

The invention further provides a KTAC compound having the structure of formula I:

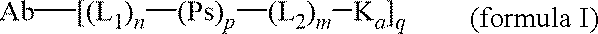

wherein Ab is a monoclonal or polyclonal antibody/antibody fragment having a binding site for a tissue specific antigen or a binding site for an antigen overexpressed in a disease or condition treatable by the monoclonal or polyclonal antibody/antibody fragment. L₁ and L₂ are linkers wherein either, neither, or both contain an acid-labile moiety site; Ps is a linker that includes at least one protease cleavage site, and Kₐ includes a kappa opioid receptor agonist peptide, thereby also releasably providing the Ka moiety that includes the kappa opioid receptor peptide agonist in addition to the therapeutic monoclonal or polyclonal antibody/antibody fragment, Ab, at the target site.

In formula I: (1) n and m are each independently 1 or zero, corresponding to presence or absence of Li and L₂, respectively; (2) p is zero or an integer from 1 to about 10, that is corresponding to absence of the peptide Ps when p = 0, or to from one to about ten copies of Ps when p is an integer; (3) q is an integer from 1 to about 10, corresponding to from one to about ten copies of —[(L₁)ₙ—(Ps)ₚ—(L₂)ₘ—Kₐ]_{q} per (KTAC) conjugate molecule, arrayed monomerically on different locations of Ab; (4) alternatively, r copies, where r is an integer from 1 to about 10, of —[(L₁)ₙ—(Ps)ₚ—(L₂)ₘ—Kₐ] can be covalently linked to form a linear polymer —[(L₁)ₙ—(Ps)ₚ—(L₂)ₘ—Kₐ]ᵣ, with from one to about ten copies of —[(L₁)ₙ—(Ps)ₚ—(L₂)ₘ—Kₐ]_{rq} per (KTAC) conjugate molecule, arrayed as monomers at different locations of Ab.

In the KTAC compound of formula I, the antibody, antibody fragment, receptor, or receptor fragment Ab is covalently bound to Ps via linker component -(L₁)ₙ- when n = 1, or directly to (Ps)ₚ when n= 0, corresponding to the absence of L₁.

In the KTAC compound of formula I, the moiety Kₐ is covalently bound to (Ps)ₚ through (L₂)ₘ when m = 1, or directly to (Ps)ₚ when m = 0, corresponding to the absence of L₂.

The invention further provides an acid-labile KTAC compound having the structure of formula I, wherein the linker (L₁)ₙ—(Ps)ₚ—(L₂)ₘ is hydrolyzed under acidic conditions. In the event that p is zero (i.e., when the peptide that includes at least one protease cleavage site is absent), then n plus m is greater than or equal to 1 (i.e., one or both of (L₁)ₙ and (L₂)ₘ is present), and at least one of (L₁)ₙ and (L₂)ₘ includes an acid-labile moiety (ALM).

Further, when n and m are each zero, i.e., when (L₁)ₙ and (L₂)ₘ are both absent, then p is 1, meaning that in this condition, the peptide containing at least one protease cleavage site, (Ps) is necessarily present as in this embodiment no ALM is present.

In one embodiment, the KTAC compound having the structure of formula I includes a kappa opioid receptor agonist peptide, Kₐ, which itself includes one, two, three, four or five D-amino acids. In one example of such an embodiment, the KTAC compound includes a D-amino acid tetrapeptide, i.e., all four amino acids are D-amino acids. In a further example, the D-amino acid tetrapeptide is a D-amino acid tetrapeptide amide such as any of the D-amino acid tetrapeptide amides disclosed in U.S. Pat. Nos. 5,965,701 of Junien et al., 7,713,937 Schteingart et al., and 10,550,150 of Desai et al., as well as those disclosed by Hughes et al., Development of a peptide-derived orally-active kappa-opioid receptor agonist targeting peripheral pain. The Open Med. Chem. J., 2013, 7,16-22. In one example, the D-amino acid tetrapeptide amide is D-Phe-D-Phe-D-Nle-D-Arg-NH-4-picolyl, known as CR665, and is disclosed in U.S. Pat. No. 5,965,701. In another embodiment, the D-amino acid tetrapeptide amide is D-Phe-D-Phe-D-Leu-D-Lys-[ω(4-aminopiperidine-4carboxylic acid)]-OH, disclosed in U.S. Pat. No. 7,713,937, also known as CR845 or difelikefalin (D-Phe-D-Phe-D-Leu-D-Lys-ɣ(4-N-piperidinyl)amino carboxylic acid]-OH) in clinical submissions or publications. In still another embodiment, the D-amino acid tetrapeptide amide is a D-Phe-D-Phe-D-Leu-D-Lys-indolylcyclopentalone or a D-Phe-D-Phe-D-Leu-D-Lys-bridged piperidine or a D-Phe-D-Phe-D-Leu-D-Lys-bridged piperazine disclosed in U.S. Pat. No. 10,550,150. In another embodiment, the D-amino acid tetrapeptide amide can be any of the above listed compounds having an N,N-dimethyl-D-Lys at the fourth position, as disclosed in Hughes et al. cited above.

The invention further provides a pharmaceutical composition including a KTAC compound having the structure of formula I and a pharmaceutically acceptable excipient; wherein formula I is Ab—[(L₁)ₙ—(Ps)ₚ—(L₂)ₘ—Kₐ]_{q} ; and Ab is an antigen binding protein or fragment that has a binding site for an antigen that is enriched in a tissue and/or present in excess in a disease or condition. The moiety Kₐ includes a kappa opioid receptor peptide agonist. The antigen binding protein or fragment Ab is covalently bound to the moiety Kₐ, including the kappa opioid receptor peptide agonist, through the linker (L₁)ₙ—(Ps)ₚ—(L₂)ₘ.

The KTAC compound having the structure of formula I is also useful for the treatment of patients suffering from kappa opioid receptor-associated diseases and conditions such as pain, inflammation, and pruritus.

### Monoclonal Antibodies

The immunoglobulin G molecule is composed of two light and two heavy chains, bound together by noncovalent interactions as well as several disulfide bonds and the light chains are disulfide-bonded to the heavy chains in the CL and CH regions, respectively. The heavy chains are in turn disulfide-bonded to each other in the hinge region.

The heavy chains of each immunoglobulin molecule are identical. Depending on the class of immunoglobulin, the molecular weight of these subunits ranges from about 50,000 to around 75,000. Similarly, the two light chains of an antibody are identical and have a molecular weight of about 25,000. For IgG molecules, the intact molecular weight representing all four subunits is in the range of 150,000-160,000.

There are two forms of light chains that may be found in antibodies..A single antibody will have light chain subunits of either lambda (λ) or kappa (κ) variety, but not both types in the same molecule. The immunoglobulin class, however, is determined by an antibody’s heavy chain variety. A single antibody also will possess only one type of heavy chain (designated as γ, µ, α, ε, or δ). Thus, there are five major classes of antibody molecules, each determined from their heavy chain type, and designated as IgG, IgM, IgA, IgE, or IgD. Three of these antibody classes, IgG, IgE, and IgD, consist of the basic lg monomeric structure containing two light and two heavy chains. By contrast, IgA molecules can exist as a singlet, doublet, or triplet of this basic Ig monomeric structure, while IgM molecules are large pentameric constructs. Both IgA and IgM contain an additional subunit, called the J chain-a very acidic polypeptide of molecular weight 15,000 that is very rich in carbohydrate. The heavy chains of immunoglobulin molecules also are glycosylated, typically in the CH2 domain within the Fc fragment region, but also may contain carbohydrate near the antigen binding sites.

There are two antigen binding sites on each of the basic Ig-type monomeric structures, formed by the heavy-light chain proximity in the N-terminal, hypervariable region at the tips of the “y” structure. The unique tertiary structure created by these subunit pairings produces the conformation necessary to interact with a complementary antigen molecule. The points of interaction on the immunoglobulin molecule with may encompass numerous non-sequential amino acids within the heavy and light chains. The binding site is formed not strictly from the linear sequence of amino acids on each chain, but from the unique orientation of these groups in 3D space. The binding site thus has affinity for a particular antigen molecule due to both structural complementarity as well as the combination of van der Waals, ionic, hydrophobic and hydrogen bonding forces bonding forces which may be created at each point of contact.

Generally useful enzymatic derivatives of antibody molecules may be prepared that still retain the antigen binding activity. Enzymatic digestion with papain produces two small fragments of the immunoglobulin molecule, each containing an antigen binding site (Fab fragments), and one larger fragment containing only the lower portions of the two heavy chains (Fc, “fragment crystallizable”). Alternatively, pepsin cleavage produces one large fragment containing two antigen binding sites [F(ab′)₂] and many smaller fragments formed from extensive degradation of the Fc region. The F(ab′)₂ fragment is held together by retention of the disulfide bonds in the hinge region. Specific reduction of these disulfides using 2-mercaptoethylamine (MEA) or other suitable reducing agents produces two Fab′ fragments, each with one antigen binding site.

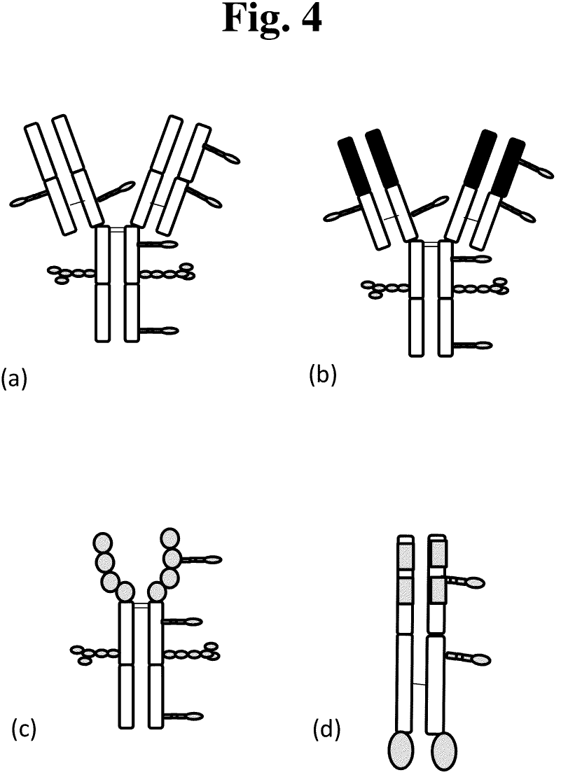

There are many available monoclonal antibodies useful in the practice of the present invention, including the following therapeutic antibodies and fragments thereof: Adulimumab is a fully humanized monoclonal anti-TNF-alpha antibody. The PEGylated IgG1 Fab′, certolizumab is a humanized Mab fragment that neutralizes TNF-alpha with high affinity. Adulimumab and certolizumab (See FIG. 4) are used in the treatment of diseases and conditions in which inflammation plays a major role, including moderate-to-severe rheumatoid arthritis, psoriatic arthritis, ankylosing spondylitis, and Crohn’s disease. Abrezekimab (TNX-650) is a humanized anti-IL13 monoclonal antibody used in the treatment of refractory Hodgkin’s Lymphoma.

Other currently approved therapeutic antibodies include secukinumab, ixekizumab and ustekinumab. Secukinumab is a fully humanized IgG1 Mab that specifically binds the cytokine IL-17A. Ixekizumab is a high affinity humanized IgG4 Mab that specifically targets IL-17A. Secukinumab and Ixekizumab are approved for use in the treatment of plaque psoriasis, psoriatic arthritis, and ankylosing spondylitis. Ustekinumab is a humanized Mab that antagonizes IL-12 and IL-23, and is FDA-approved for use in treatment of psoriasis.

Other therapeutic Mabs suitable for incorporation in the comjugates of the present invention include anti-interleukin-1 (anti-IL-1) receptor Mabs, anti-IL-6 receptor Mabs (such as tocilizumab), anti-α4 integrin subunit Mabs, and anti-CD20 Mabs. Many of these Mabs have been shown to be efficacious in clinical trials and have been approved for the therapy of several inflammatory and immune diseases and conditions, including rheumatoid arthritis, Crohn’s disease, ulcerative colitis, spondyloarthropathies, juvenile arthritis, psoriasis, and psoriatic arthritis. These novel biologics have been used as monotherapies or in combination with other therapies, especially when the disease or condition being treated is refractory to conventional therapies.

Further examples of therapeutic antigen-binding proteins useful in the practice of the present invention include receptor-binding domains linked to the immunoglobulin frame as a stabilized fusion protein, such as, for instance, etanercept, which is a fully humanized dimeric fusion protein consisting of the human Fc portion of IgG1 linked to the extracellular ligand-binding domain of the TNF-alpha p75 receptor, produced using recombinant DNA technology. The therapeutic antigen binding protein of the Ab of the invention can be modified to form a fusion protein consisting of the human Fc portion of IgG1 linked to the extracellular ligand-binding domain of a cell surface receptor for any proinflammatory cytokine, as in etanercept® shown in FIG. 4(c).

Etanercept, like these other TNF-alpha inhibitors, has been used clinically for the treatment of inflammatory conditions such as rheumatoid arthritis, ankylosing spondylitis, psoriatic arthritis, and psoriasis, among other indications. Another therapeutic antigen-binding protein useful in the practice of the present invention, anakinra, is a modified form of the endogenous IL-1 receptor antagonist that binds cell surface IL-1 receptors without activating them, thus preventing activation by the pro-inflammatory cytokine, IL-1, and is used to treat rheumatoid arthritis.

### Proteases

Proteases are enzymes that hydrolyze peptide bonds within endogenous substrates and peptides, but can also act on exogenously administered peptides and proteins. They play a key role in regulating many physiological conditions, and protease activity is dysregulated in many diseases, including inflammatory disorders and conditions with an inflammatory component. As reviewed elsewhere (see for instance, Kasperkiewicz et al., Toolbox of fluorescent probes for parallel imaging reveals uneven location of serine proteases in neutrophils J. Am. Chem Soc. 2017 vol. 139 (29) 10115-10125), five major families of proteases have been described: serine, cysteine, metallo-, aspartyl and threonine proteases. The most abundant and explored families are the serine and cysteine proteases, named after the reactive nucleophilic groups in their active sites - the hydroxyl group in serine and thiol group in cysteine. Mechanistically, proteases hydrolyze peptide bonds within the substrate (endopeptidases) or at the N or C termini (exopeptidases). Proteinases belonging to the same family, such as caspases, neutrophil serine proteases, aminopeptidases, cathepsins or kallikreins, typically have similar functions and process the same naturally occurring substrates.

To measure changes in the activity of proteolytic enzymes in cells or even in whole organisms in order to explore the individual physiological and pathophysiological functions of proteases, investigators have devised and employed various chemical tools, including substrates, inhibitors, and activity-based probes (ABPs). However, one of the greatest challenges in the design and testing of substrates, inhibitors and ABPs is achieving specificity toward only one enzyme, as the cross-reactivity of these compounds with other enzymes can significantly impair their utility. Accordingly, the specificity of the substrates, inhibitors and ABPs for proteolytic enzymes is optimized by selecting the appropriate amino acid sequences that interact with the binding pockets of the protease. There is now substantial information on different protease substrate specificities based on the development and application of multiple methods, including positional scanning synthetic combinatorial libraries, phage display, hybrid combinatorial substrate libraries, counter selection substrate libraries, internally quenched fluorescent substrate libraries (also called fluorescence resonance energy transfer libraries) and proteomics (Kasperkiewicz et al., supra). The amino acid sequence motifs thereby identified are frequently used as a starting point in the design of specific active-site directed protease inhibitors (Drag and Selvesen, Emerging principles in protease-based drug discovery, Nature Reviews Drug Discovery 2010 vol. 9, 690-701), but can also be used to design cleavage sites to be incorporated into peptide-protein conjugates, as disclosed, for example, in US10,441,649. By way of non-limiting examples, as disclosed in US10,441,649, cathepsin B cleaves at the dipeptide sequences FR, FK, VA and VR amongst others; cathepsin D cleaves the peptide sequence PRSFFRLGK; ADAM28 cleaves the peptide sequences KPAKFFRL, DPAKFFRL, KPMKFFRL, and LPAKFFRL; and the matrix metalloproteinase MMP2 cleaves the peptide sequence AIPVSLR.

### The Protease Cleavable Peptide (Ps)

In one embodiment, the Ps peptide of the KTAC compound having the structure of formula I, includes a protease cleavage site linking the antibody/antibody fragment Ab to the kappa opioid agonist, wherein the protease cleavage site is cleavable by a tissue-specific protease. According to one method of the invention, a practitioner skilled in the art utilizes the results of clinical studies to identify a protease (or proteases) that are relatively enriched or exhibit elevated activity in tissues in which a disease- or disorder-related inflammatory process is occurring, and then selects, based on the published (or otherwise available) information about the substrate specificity of said protease (or proteases), a substrate sequence that is most suitable for incorporation into a KTAC molecule as a Protease Cleavable Peptide (Ps). The practitioner employs criteria for suitability well known to those skilled in the art, including the selection of substrate sequence(s) that minimize cross-reactivity (e.g., <10%, preferably <1%, more preferably <0.1%, and most preferably <0.01%) with proteases outside of the target area, where the target area is defined as tissues in which a disease- or disorder-related inflammatory process is occurring and/or characteristically associated with the disease or disorder which the KTAC is being designed to treat.

The Ps peptide contains or consists of a protease cleavage site which, upon cleavage, functions to release the kappa opioid receptor agonist peptide in an active form from the Ab, which serves as both a targeting moiety for this peptide and as a co-therapeutic. In some embodiments, the KTAC compound may incorporate multiple copies of —[(L₁)ₙ—(Ps)ₚ—(L₂)ₘ—Kₐ]_{q}, i.e. where q>1, and p>1, such that more than one type of protease cleavage site is present in the plurality (q) of —[(L₁)ₙ—(Ps)ₚ—(L₂)ₘ—Kₐ] moieties coupled to Ab.

In another embodiment, the protease capable of cleaving the protease cleavage site is selected from the group consisting of a neutral protease, a serine protease, a cysteine protease, and a matrix metalloprotease. The protease cleavage site can be any suitable protease cleavage site such as, for instance, a protease cleavage site cleavable by a proteases such as chymotrypsin, trypsin, tryptase, subtilisin, signal peptidase, matrix metalloprotease 1, matrix metalloprotease 2, matrix metalloprotease 3, matrix metalloprotease 7, chymases (e.g., mast cell protease, skeletal muscle protease and skin protease) and neutral proteases (e.g., bacillolysin and dispase).

The protease cleavage site in the (Ps)ₚ linker can be any suitable endopeptidase cleavage site, such as a protease cleavage site cleavable by a neutral protease, a serine protease or a matrix metalloproteinase. The neutral protease can be any suitable neutral protease, such as, for instance, bacillolysin or dispase. The serine protease can be any of the many serine proteases, such as, for instance, and without limitation, mast cell serine protease, a chymase (e.g., mast cell protease, skeletal muscle protease or skin protease), kallikreins (e.g., hK1-hK15), chymotrypsin and chymotrypsin-like neutrophil serine proteases (e.g., neutrophil elastase, cathepsin G, proteinase 3, and neutrophil serine proteinase 4), trypsin, tryptase, matriptase (which is activated by exposure to acidic pH, e.g., as it occurs in skin), subtilisin, or a signal peptidase. The cysteine protease can be any suitable cysteine protease (e.g., a caspase or a paracaspase, or a cysteine cathepsin, such as cathepsins L, V, K, S, F, and B). The matrix metalloproteinase can be any suitable matrix metalloproteinase (e.g., among the 23 members of the zinc-dependent endopeptidase family in the metzincin class of metalloendopeptidases that share a common domain structure, most of which fall into one of four traditional groups of MMPs: collagenases, gelatinases, stromelysins and membrane-type MMPs.

In some embodiments, the KTAC compound may incorporate more than one type of protease cleavage site in the (Ps)ₚ linker, e.g., at different sites of attachment of a linker, (L₁)ₙ—(Ps)ₚ—(L₂)ₘ, linking the antigen-binding protein (e.g., antibody/antibody fragment) Ab to the kappa opioid agonist of the KTAC compound.

In one embodiment, the KTAC compound includes a linker, (L₁)ₙ—(Ps)ₚ—(L₂)ₘ (in which n>0, p=0, and m=0), linking the Ab to the kappa opioid agonist of the KTAC compound having the structure of formula I, wherein the linker includes an acid labile moiety (ALM) hydrolysable under acidic conditions. In other embodiments, the KTAC compound may incorporate one type or more than one type of protease cleavage site in the (Ps)ₚ linker, in addition to a linker that includes an ALM, said linkers being attached at different sites on Ab, thereby enabling the kappa opioid agonist of the KTAC compound to be released in a tissue containing the corresponding protease(s) and/or an acidic microenvironment.

A practitioner skilled in the art will design and synthesize a KTAC compound in accordance with its intended therapeutic use based on the principles as disclosed herein, particularly with respect to the selection of protease cleavage sites and/or acid-cleavable sites that are consistent with the presence of specific proteases and/or acidic conditions in a tissue of therapeutic importance in a particular inflammatory disease or inflammation-associated condition. For example, it is known that skin pH in atopic dermatitis patients is often increased into the neutral to basic range (Panther and Jacob, 2015 The importance of acidification in atopic eczema: an underexplored avenue for treatment. J. Clin. Med. 4(5), 970-978), so a KTAC compound designed to treat atopic dermatitis would not contain an acid-labile site alone, but instead incorporate a Ps with a substrate sequence that was selected based on the known substrate selectivity of a protease with elevated activity in the skin of such patients. For example, KLK5, a trypsin-like serine protease, and KLK7, a chymotrypsin-like serine protease, are major epidermal kallikrein-related peptidases (KLKs) that are increased in atopic dermatitis and thought to have a role in the pathogenesis of the disease (see Nomura et al., Multifaceted analyses of epidermal serine protease activity in patients with atopic dermatitis. Int. J. Mol. Sci., 2020, 21, 913); thus, for example, KLK5 and/or KLK7 substrate sequences could be among one or more sequences selected for Ps moieties/modules in a KTAC compound designed to treat atopic dermatitis. In contrast, in inflammatory bowel disease (IBD), MMPs, neutrophil elastase and cathepsins are typically overexpressed in the gut epithelium and basement membrane, and are therefore appropriate for consideration in designing a Ps with a corresponding substrate sequence for an IBD gut-targeted KTAC according to the methods disclosed herein.

The novelty, as well as certain features and advantages of the invention, may be more clearly apparent by considering that compounds of formula 1 contain three functional domains: an inflammatory tissue-targeting domain, an activating domain, and an anti-inflammatory therapeutic domain. Within the scope of the invention, a particular chemical moiety can encompass more than one functional domain, depending upon the specific design and desired functionality of the KTAC compound. For example, the linker can serve as both a tissue-targeting domain and an activating domain if the linker contains a protease cleavage site for a protease that is relatively enriched or exhibits increased activity in a tissue in which an inflammatory process is occurring. Likewise, the antigen-binding moiety can encompass more than one functional domain, e.g., an antibody moiety can serve as an inflammatory tissue-targeting domain and an anti-inflammatory therapeutic domain if the antigen is a cell surface protein that is preferentially expressed in a tissue subject to inflammation and mediates pro-inflammatory activity, such as a pro-inflammatory cytokine receptor, e.g., the TNF-alpha receptor or the IL-6 receptor. Alternately, the antigen-binding moiety can serve solely as an anti-inflammatory therapeutic domain if the antigen is a pro-inflammatory substance that is released by cells in a tissue subject to inflammation, such as a pro-inflammatory cytokine. In one embodiment of the invention, the foregoing antigen-binding moiety can be an antibody to said antigen, e.g., a TNF-alpha antibody. In another embodiment of the invention, the foregoing antigen-binding moiety can be a specific antigen-binding protein that consists of a sufficiently high-affinity span of the binding domain of an endogenous receptor for said antigen, e.g., the soluble form of the TNF-alpha receptor or other TNF-alpha binding protein. Further, it is envisaged that the kappa opioid agonist moiety will primarily serve as an anti-inflammatory therapeutic domain, becoming fully active only following cleavage from the activating domain linker, whether mediated by a protease or an acidic microenvironment. One advantage of this embodiment of the invention is preferential delivery of the kappa opioid agonist to the site of inflammation, thereby increasing its efficacy and reducing the likelihood of side effects due to interaction of the kappa opioid agonist with receptors in non-inflamed tissues that are therapeutically irrelevant. Moreover, in another embodiment of the invention, the linker comprises only D-amino acids to preclude protease/peptidase digestion, and the length of the linker extended to facilitate interaction of the uncleaved kappa opioid agonist moiety with cell surface kappa opioid receptors, in which case the antigen-binding moiety serves as the primary inflammatory tissue-targeting domain, and additionally as an anti-inflammatory therapeutic domain if the antigen is a cell surface protein that is preferentially expressed in a tissue subject to inflammation and mediates pro-inflammatory activity, such as a pro-inflammatory cytokine receptor, e.g., the IL-6 receptor. In this embodiment of the invention, the uncleaved kappa opioid agonist moiety retains sufficient agonist activity and affinity for kappa opioid receptors to serve as an anti-inflammatory therapeutic domain, and secondarily as a co-targeting domain with the attached antigen-binding moiety.

The invention also provides a method of treating a disease or condition, wherein the method includes administering to a patient in need thereof an effective amount of a KTAC compound having the structure: Ab—[(L₁)ₙ—(Ps)ₚ—(L₂)ₘ—Kₐ]_{q} of formula I and thereby treating the disease or condition.

In one example of this embodiment the invention provides a method of treating a disease or condition, wherein the method includes administering to a patient in need thereof an effective amount of the conjugate molecule having the structure:

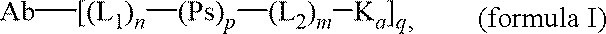

such as for instance, a conjugate molecule having the structure of formula I, wherein the kappa opioid receptor agonist component, Kₐ, is the D-amino acid tetrapeptide amide D-Phe-D-Phe-D-Leu-D-Lys-[ω (4-aminopyperidine-4carboxylic acid)]-OH, also known as CR845 (difelikefalin).

In one embodiment, the disease or condition treatable by administration of the KTAC compound having the structure: Ab—[(L₁)ₙ—(Ps)ₚ—(L₂)ₘ—Kₐ]_{q} of formula I, is a disease or condition that includes inflammation. In another embodiment the inflammatory disease or condition includes inflammation and also pruritus, interchangeably referred to herein and in the patent and non-patent scientific and medical literature with the alternate spelling, “pruritis.”

The invention further provides a pharmaceutical composition that includes a KTAC compound having the structure: Ab—[(L₁)ₙ—(Ps)ₚ—(L₂)ₘ—Kₐ]_{q} of formula I and a pharmaceutically acceptable excipient or carrier.

In one embodiment, the pharmaceutical composition that includes a conjugate molecule (KTAC) having the structure: Ab—[(L₁)ₙ—(Ps)ₚ—(L₂)ₘ—Kₐ]_{q} of formula I, wherein the kappa opioid receptor agonist peptide, Ka, includes the D-amino acid tetrapeptide amide, is:

D-Phe-D-Phe-D-Leu-D-Lys-[ω (4-aminopyperidine-4carboxylic acid)]-OH, also known as CR845 (difelikefalin).

The antibody molecules or antibody fragment useful as the Ab component of the KTAC compound having the structure Ab—[(L₁)ₙ—(Ps)ₚ—(L₂)ₘ—Kₐ]_{q} of formula I, can be any suitable antibody or antibody fragment.

The five major classes of antibody molecules, immunoglobulins IgG, IgA, IgD, IgE and IgM are defined by their heavy chain type. IgG, IgD, and IgE consist of an immunoglobulin monomeric structure containing two light and two heavy chains held together by inter-chain and intra-chain disulfide bonds and non-covalent interactions. The two light chains are comprised of identical lambda (λ) or kappa (κ) chains, each having a constant region (C_{L}) and a variable region (V_{L}), with a molecular weight of about 25,000. In humans, lambda chains occur approximately twice as frequently as kappa chains. The two heavy chains of the immunoglobulins are identical within classes and have molecular weights in the range of from about 50,000 to about 75,000. Each of the two heavy chains has a constant region consisting of three distinct regions (C_{H}1, C_{H}2 and C_{H}3) and a variable region (V_{H}). The hypervariable N-terminal portion of the variable region of a light chain and the hypervariable N-terminal portion of the variable region of a heavy chain together form an antigen binding site; thus, each complete immunoglobulin molecule has two antigen binding sites. The heavy chains of IgG molecules are glycosylated, typically in the C_{H}2 domain within the Fc fragment region, as illustrated in FIG. 1, and also may be glycosylated close to the antigen binding sites. Intact immunoglobulin molecules consisting of two light and two heavy chains have a total molecular weight of between 150,000 and 160,000.

IgA molecules are found as monomers, dimers and trimers of the immunoglobulin monomer, whereas IgM molecules are immunoglobulin pentamers. IgA and IgM complexes each include an additional J chain, a glycosylated acidic polypeptide of molecular weight 15,000 that non-covalently binds the monomers of the multiplexes together.

Immunoglobulin molecules treated with papain or pepsin yield fragments that retain the antigen binding site and can be used in place of the intact molecule in the conjugates of the present invention.

Papain digestion produces two F(ab) fragments containing the antigen binding site and an Fc fragment from the C-terminal portion of the heavy chains held together by weak forces as papain digests the hinge region that includes the disulfide bonds that covalently bind the two heavy chains together.

Pepsin, by contrast, leaves the hinge region intact and digests the C-terminal portions of the heavy chains, leaving a single F(ab′)₂ fragment with both antigen binding sites intact and the two F(ab) fragments joined by a disulfide bond.

The KTAC compounds of the invention can be targeted to a specific tissue or inflammatory site by the antibody (Ab) incorporated in the conjugate. The antibody can be a monoclonal antibody or a monoclonal antibody fragment that binds a tissue specific antigen or an antigen that is overexpressed in a disease or condition, such as an inflammatory marker antigen, such as tumor necrosis factor-alpha (TNF-alpha) or a matrix metalloprotease (MMP). In addition, the monoclonal antibody or antigen-binding fragment thereof can be an activatable antibody, in which Ab is coupled to a masking moiety (MM) via a cleavable moiety (CM) that includes a substrate for a protease, such that coupling of the MM to Ab reduces the ability of Ab to bind to its cognate antigen, as disclosed in U.S. Pat. Application 2017/0096489. The activatable antibody can be bispecific, such that when activated, specifically binds to two antigen targets, as disclosed in U.S. Pat. Application 2019/0135943. The substrate sequence for CM can be the same sequence selected for Ps in the (L₁)ₙ—(Ps)ₚ—(L₂)ₘKₐ peptide, such that the same inflammation-related protease activates the therapeutic antibody and releases the kappa opioid receptor agonist in an active form. In these embodiments, each linker, containing either Ps or CM(s), can serve as both a tissue-targeting domain and an activating domain, since each linker contains a protease cleavage site for a protease that is relatively enriched and/or exhibits increased activity in a tissue in which an inflammatory process is occurring, referred to herein as an “inflammatory protease”.

### Release of Kappa Receptor Agonist and Antibody in Situ by an Inflammatory Protease or Acidic Microenvironment

Conjugation of the kappa opioid receptor agonist to an antibody through a linker that includes a protease cleavage site and/or an acid-labile site can be directed to specific sites on the antibody molecule to provide the KTAC compound of the invention. For example, groups of such directed specific conjugation sites that can be used to covalently bind the linker and kappa opioid receptor agonist complex, —[(L₁)ₙ—(Ps)ₚ—(L₂)ₘKₐ], include the naturally occurring ε-amino groups of lysine and the carboxylate groups of the glutamic acid and aspartic acid residues as well as the C-terminal carboxylate in the light and heavy chains. Conjugation to the N-terminal amino groups of the light and heavy chains, however, is more likely to affect the antigen binding activity of the conjugate molecule and so is usually less favored. In another embodiment, the purpose of said conjugation is to provide a masking moiety (MM) via a cleavable moiety (CM) that includes a substrate for a protease, such that the antibody so conjugated serves as an activatable antibody, as described above.

Another group of directed specific conjugation sites is provided by the inter-chain and intra-chain disulfide groups. These disulfide groups can be oxidized to provide reactive sulfhydryl groups for conjugation.

A third group of specific conjugation sites to which the conjugation can be directed is the polysaccharide found on antibodies produced by mammalian cells in vivo or in vitro. Since the major glycosylation occurs in the Fc region, conjugation to the aldehyde groups produced by a mild oxidizing agent such as sodium periodate is less likely to interfere with antigen binding and provides a multiplicity of sites that can be activated at will by controlling the oxidation reaction to produce the desired number of aldehydes on the polysaccharide chains.

Any suitable chemistry can be used to link the kappa opioid agonist (Kₐ) via the linker of the invention, (L₁)ₙ—(Ps)ₚ—(L₂)ₘ to specific sites on the antigen binding protein or antibody molecule (Ab). Several chemical conjugation schemes to provide the conjugates of the invention are provided below as examples only and are not intended to be taken as limiting.

### ANTIBODY CONJUGATION

Monoclonal antibodies useful for incorporation into the antigen binding protein (Ab) of the KTAC of the invention are purified by affinity chromatography using immobilized antigen or an immobilized immunoglobulin binding protein (such as protein A) prior to undergoing conjugation. Many Mabs that can be purified with intact antigen binding properties are generally also stable enough to withstand chemical modification. However, sometimes a particular Mab is partially or completely inactivated through the modification reaction. This activity loss may be avoided by physically blocking the antigen binding sites during conjugation. In other cases, conformational changes in the complementarity-determining regions are the cause of the problem. If the antigen binding is merely being blocked, then choosing an appropriate site-directed chemistry may solve the problem. On the other hand, some Mabs are too labile to undergo modification reactions, regardless of the coupling method.

The structural characteristics of many antibody molecules provide a several choices for modification and conjugation schemes (Roitt, 1977; Goding, 1986; Harlow and Lane, 1988a b, c). The chemistry used to effect conjugate formation should be chosen to yield the best possible retention of antigen binding activity.

Antibody molecules possess a number of functional groups suitable for modification or conjugation purposes. Crosslinking reagents may be used to target lysine ε-amine and N-terminal α-amine groups. Carboxylate groups also may be coupled to another molecule using the C-terminal end as well as aspartic acid and glutamic acid residues. Although both amine and carboxylate groups are as plentiful in antibodies as they are in most proteins, the distribution of them within the three-dimensional structure of an immunoglobulin is nearly uniform throughout the surface topology. Conjugation procedures that utilize these groups crosslink randomly to most parts of the antibody molecule.

Conjugation chemistry done with antibody molecules generally is more successful at preserving activity if the functional groups utilized are present in limiting quantities and only at discrete sites on the molecule. Such “site-directed conjugation” schemes make use of crosslinking reagents that can specifically react with residues that are only in certain positions on the immunoglobulin surface, usually chosen to be well removed from the antigen binding sites. By proper selection of the conjugation chemistry and knowledge of antibody structure, the immunoglobulin molecule can be oriented so that its bivalent binding potential for antigen remains available.

Two such site-directed chemical reactions are especially useful. The disulfides in the hinge region that hold the heavy chains together can be selectively cleaved with a reducing agent (such as MEA, DTT, or TCEP) to create two half-antibody molecules, each containing an antigen binding site (Palmer and Nissonoff, 1963; Sun et al., 2005). Alternatively, smaller antigen-binding fragments can be produced from pepsin and similarly reduced to form Fab′ molecules. Both of these preparations contain exposed sulfhydryl groups which can be targeted for conjugation using thiol-reactive probes or crosslinkers. Conjugations done using hinge area-SH groups will orient the attached protein or other molecule away from the antigen binding regions, thus preventing blockage of these sites and preserving activity.

The second method of site-directed conjugation of antibody molecules takes advantage of the carbohydrate chains typically attached to the CH2 domain within the Fc region. Mild oxidation of the polysaccharide sugar residues with sodium periodate generates aldehyde groups. A crosslinking or modification reagent containing a hydrazide functional group then can be used to target specifically these aldehydes for coupling to another molecule such as the (L₁)ₙ—(Ps)ₚ—(L₂)ₘ or the linked kappa receptor agonist (L₁)ₙ—(Ps)ₚ—(L₂)ₘ—Kₐ. Directed conjugation through antibody carbohydrate chains thus avoids the antigen-binding regions while allowing for use of intact antibody molecules.

Antibodies of polyclonal origin (from antisera) are usually glycosylated and work well in this procedure, but other antibody preparations may not possess polysaccharide. In particular, some monoclonal antibodies may not be post-translationally modified with carbohydrate after hybridoma synthesis. Recombinant antibodies synthesized in bacteria also may be devoid of carbohydrate.

### NHS Ester-Maleimide-Mediated Conjugation

Heterobifunctional reagents containing an amine-reactive N-hydroxy succinimide (NHS) ester on one end and a sulfhydryl-reactive maleimide group on the other end generally have great utility for producing antibody-enzyme/other conjugates. Crosslinking reagents possessing these reactive groups can be used in highly controlled, multi-step procedures that yield conjugates of defined composition and high activity. Additional suitable succinimidyl crosslinkers include SMCC(succinyl-4-[N-maleimiddomethyl]cyclohexane-1-carboxylate), MBS (m-maleimidobenzoyl-N-hydroxysuccinimide ester) and GMBS (N-γ-maleimidobutyryl-oxysuccinimide ester). SMCC and its water-soluble analog, sulfo-SMCC possess the most stable maleimide functionalities and are probably the most often used. This increased stability to hydrolysis of SMCC’s hindered maleimide allows activation of either enzyme or antibody via the amine-reactive NHS ester end, resulting in a maleimide-activated intermediate. The intermediate species then is then purified from excess crosslinker and reaction by-products before mixing with the linked kappa receptor agonist ((L₁)ₙ—(Ps)ₚ—(L₂)ₘ—Kₐ) to be conjugated.

### Conjugation With Reduced Antibodies

One method of introducing sulfhydryl residues into antibody molecules for maleimide-activated enzymes is to reduce native disulfide groups in the hinge region of the immunoglobulin structure. Reduction with low concentrations of DTT (dithiotreitol), TCEP (tris(2-carboxyethyl)phosphine) or MEA (2-mercaptoethylamine.HCl) cleaves principally the disulfide bonds holding the heavy chains together, but leaves the disulfides between the heavy and light chains intact. The use of the relatively strong reductants DTT and TCEP requires only about 3.25 and 2.75 mole equivalents respectively per mole equivalent of antibody molecule to achieve the reduction of two interchain disulfide bonds between the heavy chains of a monoclonal IgG. This limited reduction strategy retains intact antibody molecules while providing discrete sites for conjugation to thiols. Using higher concentrations of reducing reagents DTT, TCEP or MEA results in complete cleavage of the disulfides between the heavy chains and formation of two half-antibody molecules, each containing an antigen binding site. Under these conditions some interchain cleavage also occurs and results in some smaller fragments being produced. Similar reduction can be done with F(ab′)₂ fragments produced from pepsin digestion of IgG molecules. Either of these reaction steps creates half antibody fragments, each containing one light and one heavy chain and one antigen binding site. The sulfhydryl groups produced by this type of reduction can be used to couple with maleimide-activated enzymes without blocking the antigen binding site.

Antibody reduction in the presence of EDTA prevents re-oxidation of the sulthydryl groups by metal catalysis. In phosphate buffer at pH 6 -7 and 4° C., the number of available thiols is found to be decreased only by about 7 percent in the presence of EDTA over a 40-hour time span. In the absence of EDTA, this sulfhydryl loss increased to 63-90 percent in the same period.

In the antibody reduction and activation protocol below, the most critical aspects are the concentration of reducing agent and EDTA in the reaction mixture. The required level of reduction of IgG occurs with 50-100 mM MEA and 1-100 mM EDTA. For DTT or TCER, the concentration of reducing agent can be lowered to a 3-fold molar excess over the amount of antibody present. The pH of the reaction can be from pH 6 to 9, with about pH 8 being optimal. The absolute concentration of antibody can vary and still yield acceptable results.

### Antibody Reduction and Activation Protocol

1. The IgG to be reduced is dissolved at a concentration of 1-10 mg/ml in 0.1 M sodium phosphate, 0.15 M NaCl, pH 7.2, containing 10 mM EDTA.

2. Add 6 mg of MEA to each ml of antibody solution. Alternatively, add DTT or TCEP to a final concentration equal to 3 mole equivalents per mole equivalent of antibody present. Mix to dissolve.

3. Incubate for 90 minutes at 37° C.

4. The reduced IgG is purified by gel filtration using a desalting resin, performing the chromatography using 0.1 M sodium phosphate, 0.15 M NaCl, pH 7.2, containing 10 mM EDTA as the buffer. To obtain efficient separation between the reduced antibody and excess reductant, the sample size is applied to the column at a ratio of no more than 5 percent sample volume to column volume. 0.5 ml fractions are collected and monitored spectrophotometrically for protein at 280 nm. Since the reducing agents typically have no absorbance at 280 nm, the elution profile also may be monitored by use of a standard protein assay method (e.g., BCA, Thermo Fisher). The BCA-copper reagent reacts with the reductants to produce a colored product. EDTA in the chromatography buffer inhibits the BCA method somewhat, but a color response to the reducing agent peak can still be obtained. A micro-method for monitoring each fraction is as follows: a. 5 ul from each fraction is collected and placed in a separate well of a microtiter plate. b. 200 µl of BCA working reagent is added. c. The mixture is incubated at room temperature or 37° C. for 15-30 minutes or until color develops.

The color response can be assessed visually or measured by absorbance at 562 nm. To assure good separation between the antibody peak and excess MEA, at least one fraction of little or no color should separate the two peaks.

5. Fractions containing antibody are pooled and immediately mixed with an amount of maleimide-activated enzyme to obtain the desired molar ratio of antibody-to-enzyme in the conjugate. Use of a 4:1 (enzyme:antibody) molar ratio or higher in the conjugation results in high-activity conjugates.

6. React for 30-60 minutes at 37° C. or 2 hours at room temperature. Alternatively, the conjugation reaction can be allowed to proceed at 4° C. overnight.

7. The conjugate is further purified from unconjugated enzyme by immunoaffinity chromatography, such as for instance by nickel-chelate affinity chromatography. For storage, the conjugate should be frozen, Iyophilized, or sterile filtered and kept at 4° C.

### Conjugation With 2-Iminothiolane-Modified Antibodies

Traut’s reagent, or 2-iminothiolane, reacts with amine groups in proteins or other molecules in a ring-opening reaction to result in permanent modifications containing terminal sulfhydryl residues. Antibodies can be modified with Traut’s reagent to create the sulfhydryl groups necessary for conjugation with a maleimide-activated enzyme. This protocol retains the divalent nature of the antibody molecule by leaving the sulfhydryl groups intact. However, since amine modification of antibodies can take place at virtually any available lysine ε-amine location, the resultant sulfhydryl groups are distributed almost randomly over the immunoglobulin structure. Conjugation through these introduced SH groups may result in a certain subpopulation of antibodies that have their antigen binding sites obscured or blocked by enzyme molecules. Typically, enough free antigen binding sites are available in the conjugate to result in high-activity complexes useful in binding procedures. The number of sulfhydryl groups created on the immunoglobulin using such thiolation procedures is more critical to the yield of conjugated enzyme molecules than the molar excess of maleimide-activated enzyme used in the conjugation reaction. Therefore, it is important to use a sufficient excess of Traut’s reagent to obtain a sufficient number of available sulfhydryl groups. See the protocol below:

### Protocol for Preparation of 2-Iminothiolane-Modified Antibodies

1. The antibody to be modified is dissolved at a concentration of 1-10 mg/ml in 0.1 M sodium phosphate, 0.15 M NaCl, pH 7.2, containing 10 mM EDTA. The high level of EDTA is to prevent metal-catalyzed oxidation of sulthydryl groups when working with serum proteins, especially polyclonal antibodies purified from antisera.

2. Solid 2-iminothiolane (Thermo Fisher) is added to this solution to give a molar excess of 20-40 x over the amount of antibody present. As the reagent reacts, it is completely drawn into solution. Alternatively, a stock solution of Traut’s reagent can be made in DMF and an aliquot added to the antibody solution (not to exceed 10 percent DMF in the final solution).

3. The mixture is reacted for 30 minutes at 37° C. or 1 hour at room temperature.

4. The thiolated antibody is purified by gel filtration using a desalting resin, performing the chromatography using 0.1 M sodium phosphate, 0.15 M NaCl, pH 7.2, containing 10 mM EDTA as buffer. To obtain efficient separation between the reduced antibody and excess reactant, the sample size applied to the column should be at a ratio of no more than 5 percent sample volume to the total column volume. 0.5 ml fractions are collected and monitored for protein at 280 nm. To monitor the separation of the second peak (excess Traut’s reagent), the BCA Protein Assay reagent (Thermo Fisher) can be used according to the procedure described in the previous section, protocol step 4.

5. The fractions containing antibody are pooled and immediately mixed with an amount of maleimide-activated enzyme to obtain the desired molar ratio of antibody-to-enzyme in the conjugate. Use of a 4:1 (enzyme:antibody) to 15:1 molar ratio in the conjugation reaction usually results in high-activity conjugates suitable for use in preparations.

6. The mixture is reacted for 30-60 minutes at 37° C. or 2 hours at room temperature. The conjugation reaction also can be performed at 4° C. overnight.

7. The conjugate is further purified from unconjugated enzyme by immunoaffinity chromatography, e.g., by nickel-chelate affinity chromatography. For storage, the conjugate should be frozen, Iyophilized, or sterile filtered and kept at 4° C.

### Conjugation With SATA-Modified Antibodies

N-Succinimidyl-S-acetylthioacetate (SATA) is a thiolation reagent that reacts with primary amines via its NHS ester end to form stable amide linkages. The acetylated sulfhydryl group is stable until deacetylated with hydroxylamine. Thus, antibody molecules can be thiolated with SATA to create the sulfhydryl target groups necessary to couple with a maleimide-activated enzyme. Using this reagent, stock preparations of SATA-modified antibodies may be prepared and deacetylated as needed. Unlike thiolation procedures which immediately form a free sulfhydryl residue, the protected sulfhydryl group of SATA-modified proteins is stable to long-term storage without degradation.

Although amine-reactive protocols, such as SATA thiolation, result in nearly random attachment over the surface of the antibody structure, it has been shown that modification with up to 6 SATAs per antibody molecule typically results in no decrease in antigen binding activity (Duncan et al., 1983 Anal. Biochem. A New Reagent... for the preparation of conjugates... 132: 68-73). Even higher ratios of SATA to antibody are possible with excellent retention of activity.

### Protocol for Preparation of SATA-Modified Antibodies

1. The antibody to be modified is dissolved in 0.1 M sodium phosphate, 0.15 M NaCl, pH 7.2, at a concentration of 1-5 mg/ml. Phosphate buffers can be at various pH values between 7.0 and 7.6. Other mildly alkaline buffers can be substituted for phosphate in this reaction, providing they don’t contain amines (e.g., Tris) or promote hydrolysis of SATA’S NHS ester (e.g., imidazole).

2. A stock solution of SATA (Thermo Fisher) is prepared by dissolving the SATA in DMF or DMSO at a concentration of 8 mg/ml in a fume hood.

3 10-40 µl of the SATA stock solution is added per ml of 1 mg/ml antibody solution. This results in a molar excess of approximately 12- to 50-fold of SATA over the antibody concentration. A 12-fold molar excess works well, but higher levels of SATA incorporation may result in more maleimide-activated enzyme molecules able to couple to each thiolated antibody molecule. For higher concentrations of antibody in the reaction medium, the amount of SATA added is increased proportionately. DMF in the aqueous reaction medium should not exceed 10 %.

4. Allow reaction to proceed for 30 minutes at room temperature.

5. The SATA-modified antibody is purified by gel filtration using desalting resin or by dialysis against 0.1 M sodium phosphate, 0.15 M NaCl, pH 7.2, containing 10 mM EDTA. Purification is not absolutely required, since the following deprotection step is done with hydroxylamine at a significant molar excess over the initial amount of SATA added. Whether a purification step is done or not, at this point, the derivative is stable and can be stored under conditions which favor long-term antibody activity (i.e., sterile filtered at 4° C., and then frozen or lyophilized).

6. The acetylated sulfhydryl groups on the SATA-modified antibody can be deprotected as follows:

a. Prepare a 0.5 M hydroxylamine (Thermo Fisher) solution in 0.1 M sodium phosphate, pH 7.2, containing 10 mM EDTA.

b. Add 100 ul of the hydroxylamine stock solution to each ml of the SATA-modified antibody. Final concentration of hydroxylamine in the antibody solution is 50 mM.

c. React for 2 hours at room temperature.

d. Purify the thiolated antibody by gel filtration on a desalting resin using 0.1 M sodium phosphate, 0.1 M NaCl, pH 7.2, containing 10 mM EDTA as the chromatography buffer. To obtain efficient separation between the thiolated antibody and excess hydroxylamine and reaction by-products, the sample size applied to the column should be at a ratio of no more than 5 percent sample volume to the total column volume. Collect 0.5 ml fractions. Pool the fractions containing protein detected by measuring the absorbance of each fraction at 280 nm.

7. Immediately mix the thiolated antibody with an amount of maleimide-activated enzyme to obtain the desired molar ratio of antibody-to-enzyme in the conjugate. Use of a 4:1 (enzyme:antibody) to 15:1 molar ratio in the conjugation reaction usually results in high-activity conjugates.

8. React for 30-60 minutes at 37° C. or 2 hours at room temperature. The conjugation reaction also may be done at 4° C. overnight.

9. The conjugate can be further purified from unconjugated enzyme by immunoaffinity chromatography or by metal-chelate affinity chromatography. For storage, the conjugate should be kept frozen, lyophilized, or sterile filtered and kept at 4° C. Stability studies may have to be done to determine the optimal method of long-term storage for a particular conjugate.

### Reductive Amination-Mediated Conjugation

Oxidation of polysaccharide residues in glycoproteins with sodium periodate provides an efficient way of generating reactive aldehyde groups for subsequent conjugation with amine- or hydrazide-containing molecules via reductive amination. Some selectivity of monosaccharide oxidation may be accomplished by regulating the concentration of periodate in the reaction medium. In the presence of 1 mM sodium periodate at approximately 0° C., the sialic acid groups (of the carbohydrate modification found on many antibodies) are specifically oxidized at their adjacent hydroxyl residues on the 7-, 8-, and 9-carbon atoms, cleaving off two molecules of formaldehyde and leaving one aldehyde group on the 7-carbon. At higher concentrations of sodium periodate (10 mM or greater) at room temperature other sugar residues will be oxidized at points where adjacent carbon atoms contain hydroxyl groups.

Most antibody molecules can be activated for conjugation by brief treatment with periodate. Crosslinking with an amine-containing protein takes place under alkaline pH conditions through the formation of Schiff base intermediate. These relatively labile intermediates can be stabilized by reduction to a secondary amine linkage with sodium cyanoborohydride.

Reductive amination crosslinking can be achieved using sodium borohydride or sodium cyanoborohydride; however cyanoborohydride is the better choice since it is more specific for reducing Schiff bases and will not reduce aldehydes. Small blocking agents such as lysine, glycine, ethanolamine, or Tris can be added after conjugation to quench any unreacted aldehyde sites. Ethanolamine and Tris are the best choices for blocking agents, since they contain hydrophilic hydroxyl groups with no charged functional groups.

### Activation of Antibodies with Sodium Periodate

Many immunoglobulin molecules are glycoproteins that can be periodate-oxidized to contain reactive aldehyde residues. Polyclonal IgG molecules often contain carbohydrate in the Fc portion of the molecule. This is sufficiently removed from the antigen binding sites to allow conjugation to take place through the polysaccharide chains without compromising activity. Occasionally, however, some antibodies may contain sites of glycosylation near the antigen binding regions, and in this situation conjugation through these sites may affect binding activity.

Oxidation of the antibody with subsequent conjugation to an amine- or hydrazide-containing molecule can be used to produce the desired conjugate of the invention. It should be noted, however, that many monoclonal antibodies are not glycosylated and therefore cannot be used in this method. Similarly, recombinant antibodies synthesized in bacteria also do not contain carbohydrate and therefore also are excluded from this conjugate production method.

### Protocol

1. The antibody to be periodate-oxidized is dissolved at a concentration of 10 mg/ml in 0.01 M sodium phosphate, 0.15 M NaCl, pH 7.2.

2. Sodium periodate is protected from light and dissolved in water to a final concentration of 0.1 M.

3. Immediately, 100 ul of the sodium periodate solution is added to each ml of the antibody solution and mixed to dissolve while protecting from light.

4. React in the dark for 15-20 minutes at room temperature.

5. The reaction is immediately quenched by the addition of sodium sulfite (Na₂SO₃) to provide a 2-fold molar excess over the initial amount of periodate added. The oxidized antibody is purified by gel filtration using a desalting resin with chromatography buffer, 0.1 M sodium phosphate, 0.15 M NaCl, pH 7.2. To obtain efficient separation between the oxidized antibody and excess periodate, the sample size applied to the column should be at a ratio of no more than 5 percent sample volume to the total column volume. Fractions of 0.5 ml are collected and monitored for protein at 280 nm.

6. Pool the fractions containing protein. Adjust the antibody concentration to 10 mg/ml for the conjugation step. The oxidized antibody should be used immediately.

### Conjugation of Periodate-Oxidized Antibodies with Amine or Hydrazide Derivatives

The following method requires that the antibody has already been periodate-oxidized by the method described above to create reactive aldehyde groups suitable for coupling with amine or hydrazide-containing molecules. This is an excellent method for directing the antibody modification reaction away from the antigen binding sites, if the antibody glycosylation points are solely in the Fc region of the molecule. It should be noted, however, that periodate-oxidized antibodies can self-conjugate through their own amines if high-pH reductive amination is used. Conjugation with periodate-oxidized antibodies works best if the receiving molecule is modified to contain hydrazide groups and the reaction is done at more moderate pH values (e.g., slightly acidic to neutral pH).

### Conjugation of Periodate-Oxidized Antibodies

1. For conjugation to hydrazide-containing proteins, the periodate-oxidized antibody is dissolved at a concentration of 10 mg/ml in 0.1 M sodium phosphate, 0.15 M NaCl, pH 6.0-7.2. For conjugation to amine-containing molecules and proteins, the oxidized antibody is dissolved to 10 mg/ml in 0.2 M sodium carbonate, pH 9.6.

2. Hydrazide-containing peptide is dissolved in 0.2 M sodium carbonate, pH 9.6.

3. The antibody solution from step 1 is mixed with the peptide solution from step 2 in amounts necessary to obtain the desired molar ratio for conjugation. Preferably, the peptide is reacted in approximately a 4- to 15-fold molar excess over the amount of antibody present.

4. React for 2 hours at room temperature.

5. In a fume hood, as cyanoborohydride is extremely toxic, 10 ul of 5 M sodium cyanoborohydride (Sigma) is added per ml of reaction solution. Any contact with the reagent should be avoided, as the 5 M solution is prepared in 1N NaOH. The addition of a reductant is necessary for stabilization of the Schiff bases formed between an amine-containing peptide and the aldehydes on the antibody. The addition of a reductant during hydrazide/aldehyde actions increases the efficiency and yield of the reaction.

6. React for 30 minutes at room temperature (in a fume hood).

7. Unreacted aldehyde sites are blocked by addition of 50ul of 1 M ethanolamine, pH 9.6, per ml of conjugation solution. Approximately a 1 M ethanolamine solution is prepared by addition of 300 ul ethanolamine to 5 ml of deionized water. Adjust the pH of the ethanolamine solution by addition of concentrated HCI, while keeping the solution cool on ice.

8. React for 30 minutes at room temperature.

9. The conjugate is purified from excess reactants by dialysis or gel filtration using desalting resin. 0.01 M sodium phosphate, 0.15 M NaCl, pH 7.0 is used as the buffer for the desalting or gel filtration. The conjugate can be further purified by removal of unconjugated enzyme by immunoaffinity or metal-chelation chromatography.

### Conjugation Using Antibody Fragments

Antibody fragments can be used in the preparation of the KTAC compound conjugates of the invention. Selected fragmentation carried out by enzymatic digestion of intact immunoglobulins can yield lower-molecular-weight molecules that retain the ability to recognize and bind antigen. Antibody fragment KTAC conjugates display less interference with various Fc binding proteins and also less immunogenicity (due to lack of the Fc region), more facile membrane penetration, and lower nonspecific binding to surfaces or membranes.

Selective enzymatic digests of IgG results in two particularly useful fragments: Fab and F(ab′)2, prepared by the action of papain and pepsin, respectively. Most specific enzymatic cleavages of IgG occur in relatively unfolded regions between the major domains. Papain and pepsin, and similar enzymes, including bromelain, ficin, and trypsin, cleave immunoglobulin molecules in the hinge region of the heavy chain pairs. Depending on the location of cleavage, the disulfide groups holding the heavy chains together may or may not remain attached to the antigen binding fragments that result. If the disulfide-bonded region does remain with the antigen binding fragment, as in pepsin digestion, then a divalent molecule is produced [Fab′)₂] which differs from the intact antibody by lack of an extended Fc portion. If the disulfide region is below the point of digestion, then the two heavy-light chain complexes that form the two antigen binding sites of an antibody are cleaved and released, forming individual dimeric fragments (Fab) containing one antigen binding site each.

Methods for producing immobilized papain or pepsin for antibody fragmentation can be found in Hermanson et al. (1992) Immobilized affinity ligand technique. Academic Press. The following protocol describes the use of pepsin to cleave IgG molecules at the C-terminal side of the inter-heavy-chain disulfides in the hinge region, producing a bivalent antigen binding fragment, F(ab′)2, with a molecular weight of about 105,000.

### Preparation of F(ab′) Fragments Using Pepsin

1. 0.25 ml of immobilized pepsin (Thermo Fisher) is equilibrated by washing with 4x 1ml of 20 mM sodium acetate, pH 4.5 (digestion buffer and the gel is suspended in 1ml of digestion buffer.

2. 1-10 mg of IgG is dissolved in 1 ml digestion buffer and add it to the gel suspension.

3. The reaction slurry is mixed in a shaker at 37° C. for 2-48 hours. The optimal time for complete digestion varies depending on the IgG subclass and species of origin. Mouse IG1 antibodies are usually digested within 24 hours, human antibodies are fragmented in 12 hours, whereas some minor subclasses (e.g., mouse 1gG2a) require a full 48-hour digestion period.

4. After the digestion is complete, 3 ml of 10 mM Tris-HCl, pH 8.0 is added to the gel suspension and the gel is then separated from the antibody solution using filtration or by centrifugation.

5. The fragmented IgG solution is added to an immobilized protein A column containing 2 ml gel (Thermo Fisher) that was previously equilibrated with 10 mM Tris-HCl, pH 8.0.

6. After the sample has entered the gel, the column is washed with 10 mM Tris-HCl, pH 8.0, while collecting 2 ml fractions. The fractions are monitored for protein by measuring absorbance at 280 nm. The protein peak eluting unretarded from the column is F(ab′)₂.

7. Bound Fc or Fc fragments and any undigested IgG can be eluted from the column with 0.1 M glycine, pH 2.8.

Similarly, immobilized papain may be used to generate Fab fragments from immunoglobulin molecules. Papain is a sulfhydryl protease that is activated by the presence of a reducing agent. Cleavage of IgG by papain occurs above the disulfides in the hinge region, creating two types of fragments, two identical Fab portions and one intact Fc fragment.

### Preparation of Fab Fragments Using Papain

1. 0.5 ml of immobilized papain (Thermo Fisher) is washed with 4 × 2 ml of 20 mM sodium phosphate, 20 mM cysteine-HCl, 10 mM EDTA, pH 6.2 (digestion buffer), and finally suspend the gel in 1.0 ml of digestion buffer.

2. 10 mg of human IgG solution is dissolved in 1.0 ml of digestion buffer and add it to the immobilized papain gel suspension.

3. Mix the gel suspension in a shaker at 37° C. for 4-48 hours. Maintain the gel in suspension during mixing. The optimal time for complete digestion varies depending on the IgG subclass and the species of origin. Mouse IgG1 antibodies are usually digested within 27 hours, whereas other mouse subclasses require only 4 hours; human antibodies are fragmented in 4 hours (IgG1 and IgG3), 24 hours (IgG4), or 48 hours (IgG2); whereas bovine, sheep, and horse antibodies are somewhat resistant to digestion and require a full 48 hours.

4, After the required time of digestion, 3.0 ml 10 mM Tris-HCl buffer, pH 8.0 is added to the gel suspension, mix, and then the digest solution is separated from the gel by filtration or centrifugation at 2,000 g for 5 minutes.

5. The supernatant liquid is then applied to an immobilized protein A column (2 ml gel; ThermoFisher) which was previously equilibrated by washing with 20 ml of 10 mM Tris-HCl buffer, pH 8.0.

6. After the sample has entered the gel bed, the column is eluted with 15 ml of 10 mM Tris-HCl buffer, pH 8.0, collecting 2.0 ml fractions. The fractions are monitored for protein by absorbance at 280 nm. The protein eluted unretarded from the column is purified Fab.

7. Fc and undigested IgG bound to the immobilized protein A column can be eluted with 0.1 M glycine-HCl buffer, pH 2.8.

Conjugation of these fragments with peptides is done using similar methods to those discussed above for intact antibody molecules. F(ab′)2 fragments can be selectively reduced in the hinge region with DTT, TCEP, or MEA using the identical protocols outlined for whole antibody molecules. Mild reduction results in cleaving the disulfides holding the heavy chain pairs together at the central portion of the fragment, creating two F(ab′) fragments, each containing one antigen binding site.

The amine groups on these fragments can also be modified with thiolating agents, such SATA or 2-iminothiolane, to create sulfhydryl residues suitable for coupling to maleimide-activated peptides.

### Immunoaffinity Chromatography

Immunoaffinity chromatography makes use of immobilized antigen molecules to bind and separate specific antibody from a complex mixture. After the preparation of an antibody-peptide KTAC conjugate, the antibody binding capability of the crosslinked complex toward its complementary antigen ideally remains intact. This highly specific interaction can be used to purify the conjugate from excess enzyme if the antibody and enzyme can survive the conditions necessary for binding and elution from such an affinity column. Binding conditions typically are mild physiological pH conditions which cause no difficulty. However, elution conditions that require acidic or basic conditions or the presence of a chaotropic agent to deform the antigen binding site can in certain cases irreversibly damage the antigen binding recognition capability of the antibody.

Another potential disadvantage of an immunoaffinity separation is the assumed abundance of the purified antigen in sufficient quantities to immobilize on a chromatography support. Protein antigens should be immobilized at densities of at least 2-3 mg/ml of affinity gel to produce supports of acceptable capacity for binding antibody. Often, the antigen is too expensive or scarce to obtain in the amounts needed. However, if the antigen is abundant and inexpensive and the antibody-peptide complex survives the associated elution conditions, then immunoafinity chromatography can provide a very efficient method of purifying a conjugate from excess reactants. This method also assures that the recovered antibody still retains its ability to bind specific target molecules (i.e., the antigen binding site was not blocked during conjugation). A suggested method for performing immunoaffinity chromatography follows.

### Immunoaffinity Chromatography Protocol

1. The immunoaffinity column is equilibrated with 50 mM Tris, 0.15 M NaCl, pH 8.0 (binding buffer) and washed with at least 5 column volumes of buffer. The amount of gel used should be based on the total binding capacity of the support. A determination of binding capacity can be done by overloading a small-scale column, eluting, and measuring the amount of conjugate that bound. Such an experiment may be coupled with a determination of conjugate viability for using immunoaffinity as the purification method. The final column size should represent an amount of gel capable of binding at least 1.5 times more than the amount of conjugate that will be applied.

2. Apply the conjugate to the column in the binding buffer while taking 2 ml fractions.

3. Wash with binding buffer until the absorbance at 280 nm decreases back to baseline. The unbound protein flowing through the column will consist of mainly unconjugated peptide. Some conjugate may flow through also if some of the conjugate is inactive or the column is overloaded.

4. Elute the bound conjugate with 0.1 M glycine, 0.15 M NaCl, pH 2.8, or another suitable elution buffer. A neutral pH alternative to this buffer is the Gentle Elution Buffer from Thermo Fisher. If acid pH conditions are used, immediately neutralize the fractions eluting from the column by the addition of 0.5 ml of 1 M Tris, pH 8.0, per fraction.

### Nickel-Chelate Affinity Chromatography

Metal-chelate affinity chromatography is a powerful purification technique whereby proteins or other molecules can be separated based upon their ability to form coordination complexes with immobilized metal ions. The metal ions are stabilized on a matrix through the use of chelating compounds which usually have multivalent points of interaction with the metal atoms. To form useful affinity supports, these metal ion complexes must have some free or weakly associated and exchangeable coordination sites. These exchangeable sites then can form complexes with coordination sites on proteins or other molecules. Substances that are able to interact with the immobilized metals will bind and be retained on the column. Elution is typically accomplished by one or a combination of the following options: (1) lowering of pH, (2) raising the salt strength, and/or (3) the inclusion of competing chelating gents such as EDIA or imidazole in the buffer.

Sorensen (1993) U.S. Pat. No. 5,266,686 disclosed that a nickel-chelate affinity column will specifically bind IgG class immunoglobulins while allowing certain enzymes to pass through the gel unretarded. This phenomenon allows the separation of antibody-conjugate complexes containing proteins or peptides conjugated to common polyclonal or monoclonal antibodies from other components. The nickel-chelate column binds the conjugate through the Fc region of the associated antibody, even if other molecules, such as the peptides of the KTAC, are covalently attached. Any unconjugated peptide will pass through the affinity column unretarded.

Elution of the bound antibody conjugate occurs by only a slight shift in pH to acidic conditions or through the inclusion of a metal-chelating agent like EDTA or imidazole in the binding buffer. Either method of elution is mild compared to most immunoaffinity separation techniques (discussed above). Thus, purification of the antibody-enzyme complex can be done without damage to the activity of either component.

One limitation to this method should be noted. If the antibody- conjugate is prepared using antibody fragments such as Fab or F(ab′)₂, then nickel-chelate affinity chromatography will not work, since the requisite Fc portion of the antibody necessary for complexing with the metal is not present.

Any metal-chelate resin designed to bind His-tagged fusion proteins also will work well in this procedure. The following protocol is adapted from the instructions accompanying the nickel-chelate support. Commercial kits are available based on this technology for the purpose of removing unconjugated reactants from such antibody conjugates.

### Nickel-Chelate Affinity Chromatography Protocol

1. Pack a column containing an immobilized iminodiacetic acid support (or another chelating agent designed to bind His-tagged proteins). The column size should be no less than 1.5 times that required to bind the anticipated amount of conjugate to be applied. The maximal capacity of such a column for binding antibody can be up to 50 mg/ml gel; however, best results are obtained if no more than 10-20 mg/ml of conjugate is applied.

2. 50 mg of nickel ammonium sulfate is dissolved per ml of deionized water and 1ml of nickel solution per ml of gel is applied to the column.

3. The column is washed with 10 volumes of water and then the support is equilibrated with 2 volumes of 10 mM sodium phosphate, 0.15 M NaCl, pH 7.0 (binding buffer).

4. The conjugate is dissolved or dialyzed into binding buffer and the conjugate solution is applied to the column while collecting 2 ml fractions.

5. Washing of the gel with 0.15 M NaCl (saline solution) is continued until the absorbance at 280 nm is down to baseline. The eluate from the column at this point is unconjugated peptide.

Additional conjugation approaches well known in the art are suitable for use in preparation of the KTAC compounds of the invention according to routine methods. See, for example, the review of Chiu et al., Antibody Structure and Function: The Basis for Engineering Therapeutics. Antibodies 2019, 8(4), 55.

### Pharmaceutical Compositions

The KTAC compound having the structure of formula I can be incorporated into pharmaceutical compositions for administration to patients in need of treatment for various inflammatory diseases and conditions.

The invention also provides a method of treating a disease or condition; the method includes administering an effective amount of a conjugate molecule having the structure of formula I to a patient in need thereof. The disease or condition can be any disease or condition involving a particular tissue where kappa opioid receptors are present, or characterized by the tissue-specific expression due to the disease or condition or relative enrichment in a tissue of one or more antigens characteristic of the disease or condition. “Relative enrichment” as used herein refers to a higher abundance or activity of an antigen in a tissue that is involved in an inflammatory process associated with a disease or condition, when compared to either (a) the same tissue in a subject when no inflammatory process is occurring, or (b) different tissues that are not involved in an inflammatory process in a subject with an inflammatory disease or condition. Relative enrichment can either be determined by measurements of a cognate antigen in biopsy samples in a patient to be treated with a KTAC, or, more practically in most medical facilities, based on prior reports of relative enrichment of said antigen in the medical or scientific literature with respect to the disease or condition being treated with a KTAC.

In one embodiment the disease or condition treatable by the method of the invention is inflammation or includes an inflammatory component. Brain and spinal tissues are generally excluded from such treatments as they are protected by the blood-brain barrier except where said barrier is compromised by a local inflammatory process, or when the KTAC is delivered by spinal administration.

Following administration to a patient with an inflammatory disease or condition including an inflammatory component, the Ps-containing KTAC compound preferentially targets active inflammatory sites in particular tissues as the degradation of the linker sequence is mediated by proteases enriched in said tissues, allowing interaction of the cleaved, free kappa opioid receptor agonist peptide with kappa opioid receptors in these tissues, as well as the release of the targeted antibody to bind to an additional inflammation-related target, thereby eliciting a combined therapeutic benefit through this unique dual mode of action. In contrast, ALM-containing KTAC compounds target active inflammatory sites based on the principle that such sites exhibit a lower pH compared to non-inflamed tissues of the same type, and therefore preferentially release the conjugated kappa opioid receptor peptide agonist from the therapeutic antigen-binding protein (Ab, e.g., a therapeutic antibody) in the inflammatory tissue acidic microenvironment. In some embodiments of the invention, certain KTAC compounds possess both Ps-linked and ALM-linked kappa opioid receptor peptide agonists conjugated to the Ab, enabling treatment of inflammatory diseases or conditions in tissues where either a specific enriched protease or an acidic microenvironment is present, or both, thereby providing increased flexibility of treatment options for a patient in need thereof. When both protease-sensitive (Ps) and acid labile moieties are present linking the Ab and the kappa opioid receptor agonist (Kₐ), said moieties can be distributed as single or multiple copies in the same or different linkers (L₁ and L₂).

### Diseases and Condition Treatable With KTACs

Diseases or conditions with an inflammatory component treatable by the methods of the invention include a wide range of disorders, including, but not limited to the following: allergy-related conditions, such as asthma, allergic contact stomatitis, allergic rhinitis, and allergies to food, drugs, toxins, dander and other known triggers of allergic responses; autoimmune diseases such as autoinflammatory syndrome, juvenile idiopathic arthritis, lupus vasculitis, rheumatoid arthritis, scleroderma, Sjogren’s syndrome, systemic lupus erythematosus, and systemic sclerosis, and undifferentiated connective tissue disease; fibromyalgia; infectious inflammatory diseases and conditions, such as acute bronchitis, the common cold, herpes simplex viral lesions, infectious mononucleosis, acute laryngitis, acute necrotizing ulcerative gingivitis, pharyngitis, laryngopharyngitis, acute sinusitis, tonsillitis, infectious osteomyelitis, cholangitis, cholecystitis, diverticulitis, endocarditis, enteritis, hepatitis, infectious arthritis such as Lyme arthritis, and poststreptococcal inflammatory syndromes, such as poststreptococcal reactive arthritis and poststreptococcal glomerulonephritis, vulvovaginitis, prostatitis, urinary tract infections, such as urethritis, cystitis and pyelonephritis; respiratory infections caused by bacteria or viruses, such as influenza or a coronavirus; inflammatory conditions of the gastrointestinal system, such as coeliac disease, Crohn’s disease, inflammatory bowel disease, irritable bowel syndrome, and ulcerative colitis; inflammatory conditions associated with surgical procedures, such as appendectomy, open colorectal surgery, hernia repair, prostatectomy, colonic resection, gastrectomy, splenectomy, colectomy, colostomy, pelvic laparoscopy, tubal ligation, hysterectomy, vasectomy or cholecystectomy, or post medical procedures, such as after colonoscopy, cystoscopy, hysteroscopy or cervical or endometrial biopsy; inflammatory conditions or inflammation-associated injuries of bones, tendons, and joints, such as adhesive capsulitis, bone fractures, bursitis, chondromalacia, chronic recurrent multifocal osteomyelitis, gout, labral tears, osteoarthritis, plantar fasciitis, pseudogout, rotator cuff tears or injuries, sesamoiditis, tendinitis (also known as tendonitis) conditions, and torn meniscus; inflammatory conditions caused by exposure to toxic agents, such as insect, plant, or jellyfish toxins, or inflammatory reactions to drugs; inflammatory conditions of the eye, such as that following photo-refractive keratectomy, ocular laceration, orbital floor fracture, chemical burns, corneal abrasion or irritation, or associated with conjunctivitis, corneal ulcers, scleritis, episcleritis, sclerokeratitis, herpes zoster ophthalmicus, interstitisal keratitis, acute iritis, keratoconjunctivitis sicca, orbital cellulites, orbital pseudotumor, pemphigus, trachoma or uveitis ; inflammatory myopathies, such as dermatomyositis, polymyositis, and inclusion-body myositis; stomatitis, such as aphthous stomatitis, chronic ulcerative stomatitis, and mucositis caused by chemotherapy, or radiation therapy of the oropharyngeal area; inflammatory conditions of the viscera, such as gastro-esophageal reflux disease, pancreatitis, acute polynephritis, glomerulonephritis, ulcerative colitis, acute pyelonephritis, cholecystitis, cirrhosis, hepatic abscess, hepatitis, duodenal or gastric ulcer, esophagitis, gastritis, gastroenteritis, colitis, diverticulitis, intestinal obstruction, ovarian cyst, pelvic inflammatory disease, perforated ulcer, peritonitis, prostatitis, interstitial cystitis; inflammatory skin conditions, such as allergic contact dermatitis or an atopic dermatitis, such as psoriasis, eczema or contact dermatitis, cutaneous drug reactions, including injection site reactions, cutaneous infections such as acne vulgaris, and acne rosacia, dermatitis herpetiformis, dermatomyositis, erythema multiforme, erythroderma, immune thrombocytopenic purpura, lichen planus, lupus ertythematosus, pemphigus disorders, prurigo nodularis, rosacia, scleroderma, seborrheic dermatitis, stasis dermatitis, and urticaria; pruritic conditions, which can be manifest in many of the foregoing inflammatory skin conditions, as well as other systemic diseases and conditions with a inflammatory component, such as end-stage renal disease, lymphoma, and chronic liver diseases, including chronic viral hepatitis B and C, cholestasis of pregnancy, primary biliary cirrhosis, Alagille syndrome, obstructive tumor in the pancreatic head, and primary sclerosing cholangitis; reperfusion injury; sarcoidosis; spondyloarthritis conditions, which have a common feature of enthesitis, such as ankylosing spondylitis, psoriatic arthritis, reactive arthritis, enthesitis-related arthritis, a form of idiopathic juvenile arthritis, and enteropathic arthritis; transplant rejection; and different forms of vasculitis, such as atherosclerosis, autoimmune vasculitis, drug-induced vasculitis, granulomatosis with polyangiitis, Henoch-Schonlein purpura, Kawasaki disease, polyarteritis nodosa, Takayasu’s arteritis, giant cell arteritis, ANCA-associated vasculitis, Buerger’s disease (thrombangiitis obliterans), and Behcet’s disease.

Many of the foregoing inflammatory conditions are relatively tissue-specific, e.g., arthritic conditions involving synovial tissues, and various forms of dermatitis and other inflammatory conditions of the skin. A practitioner skilled in the art can readily identify the tissues where inflammatory processes are occurring in these diverse conditions, and furthermore, identify the proteases which are relatively enriched in these tissues, either under baseline conditions or as a result of the inflammatory disease process.

The tissue localization and molecular characteristics of inflammatory processes, including the involvement of specific proteases in the foregoing conditions, have been intensively studied (see for instance Proteases: Multifunctional Enzymes in Life and Disease. Lopez-Otin C. and Bond, J.S. (2008) J.B.C. 283, 30433-30437), as has the tissue distribution and characteristics of different proteases. For example, their substrate specificities have been characterized (see, for example, Proteome-derived, database-searchable peptide libraries for identifying protease cleavage sites. Schilling, O. and Overall, C.M. (2008) Nature biotechnology, 26, 685-694), which is helpful in designing the protease-cleavable peptide linkers of the invention, for one skilled in the art.

Information about molecular characteristics of inflammatory processes has been used to guide the development of previous generations of anti-inflammatory therapeutics, particularly antigen binding proteins, such as antibodies, or fragments thereof, that have a binding site for particular antigens that are considered to contribute to the pathology of different inflammatory diseases or conditions. However, these targeted antigens generally have an important role in the normal functioning of the immune system, and it is widely recognized that the utility of this relatively new class of therapeutics is often constrained by dose-limiting side effects. Thus, one particular advantage of the present invention that it provides novel forms of these therapeutic agents coupled to kappa opioid agonists with complementary anti-inflammatory activities as well as inflammatory tissue targeting in order to enable the use of reduced doses of these agents, with a corresponding reduction in unwanted side effects while maintaining therapeutic efficacy.

### KTAC Compounds for the Treatment of Inflammatory Skin Diseases

In order to directly target particular KTAC compounds to sites of active inflammation, it is envisioned that specific linker sequences would be incorporated into the KTAC which are sensitive to degradation within an active inflammatory environment.

In most inflammatory diseases or conditions, mast cells are important in mediating the inflammatory process. When activated, mast cells release granules and an array of inflammatory chemical mediators into the interstitial space. These mediators include mast cell-specific proteases (tryptase and chymase), and other proteases such as cysteinyl cathepsins and matrix metalloproteinases (MMPs).

For the treatment of inflammatory skin diseases, substrate sequences specific for serine proteases that are enriched in skin-related mast cells are incorporated into the linker sequence of the KTAC compound, thereby enabling a relatively “skin-specific” degradation of the linker and release of the kappa opioid receptor agonist (Kₐ) and the therapeutic antibody (Ab) in the local inflammatory environment of the skin. This structural feature is designed to ensure delivery of therapeutic concentrations of both the kappa opioid receptor agonist and therapeutic antibody to their respective targets within the dermis and epidermis, and also to minimize systemic effects of the kappa opioid receptor agonist and therapeutic antibody.

In other embodiments of the invention, the therapeutic antibody/antibody fragment, Ab of the KTAC compound having the structure of formula I, selectively/specifically binds to an antigen overexpressed in or specific to an inflammatory tissue.

In another embodiment, the invention provides a KTAC compound having the structure Ab—[(L₁)ₙ—(Ps)ₚ—(L₂)ₘ—Kₐ]_{q} of formula I that includes an antibody/antibody fragment Ab that selectively/specifically binds to an antigen that is uniquely present, or present in excess in an inflammatory tissue or to an antigen overexpressed in a disease or condition.

In still another embodiment of the invention the KTAC compound having the structure Ab—[(L₁)ₙ—(Ps)ₚ—(L₂)ₘ—Kₐ]_{q} of formula I is a kappa opioid receptor agonist-therapeutic antibody conjugate, including a therapeutic antibody/therapeutic antibody fragment that selectively/specifically binds to an antigen specific to an inflammatory tissue or to an antigen present in excess in a disease or condition. For example, the disease or condition can be an inflammatory disease or condition. The inflammatory disease or condition can also include pruritus.

In one embodiment of the invention, a KTAC compound that includes a kappa opioid receptor agonist (Kₐ) and an IL-17 specific antibody is synthesized and used to treat patients suffering from inflammatory skin diseases or conditions, including, but not limited to, atopic dermatitis or psoriasis. In other embodiments of the invention, the foreegoing diseases and conditions can be treated by a KTAC compound of the invention wherein the Kₐ is CR845 and the Ab is an anti-IL-4 Mab, an anti-IL-17 Mab, or an anti-IL-33 Mab.

### Synthesis of (L₁)ₙ—(Ps)ₚ—(L₂)ₘ Kₐ peptide

The (L₁)ₙ—(Ps)ₚ—(L₂)ₘKₐ peptide can be produced by any suitable chemical scheme, such as by solid or liquid phase chemistry, for example, and without limitation, by the solid phase peptide synthesis described in U.S. Pat. Nos. 7,402,564, 7,713,937 and 7,842,662.

Briefly, Fmoc (fluorenylmethyloxycarbonyl) and Boc (butyloxycarbonyl) protecting groups are used to block functional groups of the amino-piperidinyl carboxylic acid in N-Boc-amino-(4-N-Fmoc-piperidinyl) carboxylic acid immobilized on a 2-chlorotrityl chloride resin and Boc and Fmoc derivatives of the D-amino acids and are used in the solid phase synthesis cycles to produce the immobilized Kₐ agonist peptide by standard procedures such as those described in the above-mentioned ‘564, ‘937 and ‘662 patents.

By way of non-limiting example, in one method of synthesis, as described in the above-mentioned ‘937 patent of Schteingart, the fully protected resin-bound Kₐ peptide portion is synthesized manually starting from a 2-chlorotrityl chloride resin. The resin is treated with Boc-4-amino-1-Fmoc-4-(piperidine)-4-carboxylic acid in a mixture of dimethylformamide (DMF), dichloromethane (DCM) and N,N-Diisopropylethylamine (DIEA). The mixture is stirred for several hours and then the resin is capped by the addition of methanol and DIEA. The resin is isolated and washed with DMF. The resin containing the first amino acid is treated with piperidine in DMF and washed several times with excess DMF. Fmoc-D-Lys(Boc)-OH is coupled to the washed resin using benzotriazole-1-yl-oxy-tris-pyrrolidino-phosphonium hexafluorophosphate (PyBOP) in the presence of hydroxybenzo-triazole (HOBt) and DIEA in DCM/DMF as solvent, stirring for several hours. The dipeptide containing resin is then isolated and washed several times with excess DMF. The Fmoc terminal protecting group is then removed by treatment with piperidine in DMF and the resin is washed with several excess volumes of DMF and treated with Fmoc-D-Leu-OH, diisopropylcarbodiimide (DIC) and HOBt in DCM/DMF and stirred for 1 hour. Subsequent washing with DMF is followed by cleavage of the Fmoc group with piperidine in DMF and then washing of the resin with DMF, providing the resin bound tripeptide. This material is treated with Fmoc-D-Phe-OH, DIC and HOBt in DCM/DMF, stirring overnight. The resin is then isolated, washed with DMF, then treated three times with piperidine in DMF to cleave the Fmoc group, and then washed again several times with DMF. The tetrapeptide-loaded resin is subsequently treated with Fmoc-D-Phe-OH, DIC, and HOBt in DCM/DMF and stirred for a few hours. The resin is then isolated, washed three times with excess DMF and treated with piperidine in DMF. The resin is then isolated, and washed sequentially with excess DMF and then with excess DCM, and dried to provide the protected Kₐ peptide bound to the resin.

Boc-L-amino acid and Fmoc-L-amino acid derivatives are then used in extending the N-terminus of the immobilized Kₐ peptide to produce the (L₁)ₙ—(Ps)ₚ—(L₂)ₘKₐ peptide immobilized on the 2-chlorotrityl chloride resin, although Boc-D-amino acid and Fmoc-D-amino acid derivatives can also be used in L₁ and L₂ linkers of the (L₁)ₙ—(Ps)ₚ—(L₂)ₘ peptide other than in the protease cleavage sites, which require L-amino acids for recognition by the cognate protease.

Acid-sensitive cleavage sites may be incorporated in the (L₁)ₙ—(Ps)ₚ—(L₂)ₘKₐ peptide by including an ester linkage or other acid-sensitive linkage in place of a peptide residue.

Any Boc-protected and Fmoc-protected L- or D-amino acids can be used in the extension of the L₂ and L₁ linkers which may function as spacer linkers, whereas Boc-protected and Fmoc-protected L-amino acids are used in the extension of the protease-sensitive Ps peptide. For example, one or more L-lysine and/or L-arginine residues may be incorporated in the Ps peptide to serve as cleavage sites for trypsin and trypsin-like proteases.

Similarly, L-tyrosine, L-phenylalanine and/or L-tryptophan residues can be incorporated in the Ps peptide to serve as cleavage sites for chymotrypsin and chymotrypsin-like proteases.

Alternatively, L-alanine, L-glycine and/or L-valine residues may be incorporated in the Ps peptide to serve as cleavage sites for elastase and elastase-like proteases. Cleavage sites for thrombin and thrombin-like proteases can be incorporated into the Ps peptide by incorporating L-arginine residues and excluding aspartic and glutamic acids from the Ps peptide as these residues prevent thrombin binding.

Metalloprotease cleavage sites can be included in the Ps peptide, for instance by incorporating the HEXXH motif, wherein H is L-histidine, E is L-glutamic acid and X can be any uncharged L-amino acid.

After completion of the extension of the full length peptide, the (L₁)ₙ—(Ps)ₚ—(L₂)ₘKₐ peptide is cleaved from the resin using trifluoroacetic acid (TFA) in water, which also serves to remove the Boc protecting groups. The mixture is filtered, concentrated and precipitated by addition to (MTBE). The solid is collected by filtration and dried under reduced pressure to give the crude (L₁)ₙ—(Ps)ₚ—(L₂)ₘKₐ peptide.

For purification, the crude peptide can be dissolved in 0.1% TFA in water and purified by preparative reverse phase HPLC (C₁₈) using 0.1% TFA/water/acetonitrile gradient as the mobile phase. Fractions with purity exceeding 95% are pooled, concentrated, and lyophilized to provide pure peptide. The peptide can be further purified by ion exchange chromatography using an ion exchange resin and eluting with water. The aqueous phase can be filtered, for instance through a 0.22 µm filter capsule, and freeze-dried to yield the purified acetate salt of the peptide:

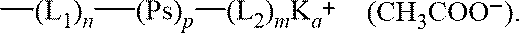

The purified peptide can then be conjugated to an activated therapeutic monoclonal antibody or antibody fragment as described above to provide a KTAC compound of the invention suitable for preclinical testing and subsequently for administration, once formulated according to standard methods well known in the art to enable provision of a therapeutic dose, selected in accordance with standard methods well known in the art, to a patient in need of treatment.

### Measuring the Potency of the Kₐ Components of the KTAC Compounds of the Invention on the Human Kappa Opioid Receptor

The compounds of the invention include conjugates of Kₐ prepared using different linkers to an Ab such that Kₐ may not, in some instances, be able to bind effectively to the human kappa opioid receptor prior to linker cleavage. Such steric inactivation of Kₐ in the conjugate can be confirmed experimentally using the method described below, and compared to the binding of the unconjugated Kₐ, e.g., CR845, or other Kₐ of the KTAC being evaluated. Steric hindrance of the bioactivity of Kₐ in a KTAC compound prior to release by linker cleavage is advantageous in limiting activity of Kₐ to the microenvironment where cleavage occurs, thereby preferentially providing active Kₐ to the targeted inflammatory tissue relative to other tissues where Kₐ actions may not provide therapeutic effects, and possibly cause undesired side effects.

Human Embryonic Kidney cells (HEK-293 cells, ATCC, Manassas, Va.) in 100 mm dishes are transfected with transfection reagent, Fugene6 (Roche Molecular Biochemicals) and DNA constructs in a 3.3 to 1 ratio. The DNA constructs used in the transfection are as follows: (i) an expression vector for the human kappa opioid receptor, (ii) an expression vector for a human chimeric G-protein, and (iii) a luciferase reporter construct in which luciferase expression is induced by the calcium sensitive transcription factor NFAT.

The expression vector containing the human kappa opioid receptor is constructed as follows: The human OPRK1 gene was cloned from human dorsal root ganglion total RNA by PCR and the gene inserted into expression vector pcDNA3 (Invitrogen, Carlsbad, Calif.) to construct human OPRK1 mammalian expression vector pcDNA3-hOPRK1.

To construct the human chimeric G-protein expression vector, the chimeric G-protein G.alpha.qi5 was first constructed by replacing the last 5 amino acids of human G.alpha.q with the sequence of the last 5 amino acids of G.alpha.i by PCR. A second mutation was introduced to this human G.alpha.qi5 gene at amino acid position 66 to substitute a glycine (G) with an aspartic acid (D) by site-directed mutagenesis. This gene was then subcloned into a mammalian expression vector pcDNA5/FRT (Invitrogen) to yield the human chimeric G-protein expression vector, pcDNA5/FRT-hGNAq-G66D-i5.

To prepare the luciferase reporter gene construct, synthetic response elements including 3 copies of TRE (12-O-tetradecanoylphorbol-13-acetate-responsive elements) and 3 copies of NFAT (nuclear factor of activated T-cells) were incorporated upstream of a c-fos minimal promoter. This response element and promoter cassette was then inserted into a luciferase reporter gene vector pGL3-basic (Promega) to construct the luciferase reporter gene plasmid construct pGL3b-3TRE-3NFAT-cfos-Luc.

The transfection mixture for each plate of cells included 6 micrograms pcDNA3-hOPRK1, 6 micrograms of pcDNA5/FRT-hGNAq-G66D-15, and 0.6 micrograms of pGL3b-3TRE-3NFAT-cfos-Luc. Cells were incubated for one day at 37° C. in a humidified atmosphere containing 5% CO₂ following transfection, and plated in opaque 96-well plates at 45,000 cells per well in 100 microliters of medium. The next day, test and reference compounds were added to the cells in individual wells. A range of concentrations of test compounds was added to one set of wells and a similar range of concentrations of reference compounds was added to a set of control wells. The cells were then incubated for 5 hours at 37° C. At the end of the incubation, cells were lysed by adding 100 microliters of detection mix containing luciferase substrate (AMP (22 ug/ml), ATP (1.1 mg/ml), dithiothreitol (3.85 mg/ml), HEPES (50 mM final concentration), EDTA (0.2 mg/ml), Triton N-101 (4 ul/ml), phenylacetic acid (45 ug/ml), oxalic acid (8.5 ug/ml), luciferin (28 ug/ml), pH 7.8). Plates were sealed and luminescence read within 30 minutes. The concentration of each of the compounds was plotted against luminescence counts per second and the resulting response curves subjected to non-linear regression using a four-parameter curve-fitting algorithm to calculate the EC₅₀ (the concentration of compound required to produce 50% of the maximal increase in luciferase activity) and the efficacy (the percent maximal activation compared to full induction by any of the well-known kappa opioid receptor agonists, such as asimadoline (EMD-61753: See Joshi et al., 2000, J. Neurosci. 20(15):5874-9), or U-69593: See Heidbreder et al., 1999, Brain Res. 616(1-2):335-8).

### Assay of Anti-inflammatory Activity in an In Vitro Model of Human Macrophage-Mediated Inflammation

Human monocyte-derived macrophages were stimulated with lipopolysaccharide (LPS) and interferon gamma (IFN-γ) in the presence and absence of CR845 or interleukin-10 (IL-10; 10 ng/mL). Cytokine release in the 18-hour supernatants was measured by multiplex immunoassay (N=4 donors per condition, with assays run in triplicate), with results expressed as mean cytokine release (± SEM) following stimulation with LPS and IFN-γ. CR845 significantly reduced the secretion of the pro-inflammatory cytokine tumor necrosis factor (TNF), interleukins-1β, -6, and -8 (IL-1β, IL-6, and IL-8), and the hormone, granulocyte colony-stimulating factor (GCSF), following stimulation of primary human macrophages with LPS and IFN-γ (***, **, * denote p < 0.001, <0.01, < 0.05 vs. vehicle, respectively; one- or two-way ANOVA). The minimum effective concentration was 2.0 nM, which was the lowest concentration tested (see Table 1). This effect was blocked by the selective kappa opioid antagonist, nor-BNI, indicating that the anti-inflammatory effects of CR845 are likely mediated via activation of the KORs localized on this population of human immune cells.

TABLE 1

Effects of CR845 on Cytokine Release from LPS/IFN Human Monocyte-Derived Macrophages

| | | No Treatment | CR845 (2 nM) | CR845 (10 nM) | CR845 (50 nM) | CR845 (50 nm) + nor-BNI (10 nM) | IL-10 | nor-BNI (10 nM) |
|---|---|---|---|---|---|---|---|---|
| | No LPS/IFNγ | LFS/IFNγ Stimulated | | | | | | |
| | Cytokine (pg/mL) | | | | | | | |
| IL-1 | 1.6 (0.0) | 75.4 (7.6) | 30.8 ^{**} (2.2) | 45.3 ^{**} (1.1) | 21.5 ^{**} (2.4) | 66.7 ^{##} (2.3) | 2.1 ⁵⁵ (1.1) | 39.9 (1.1) |
| IL-6 | 63.8 (1.2) | 677.7 (96.4) | 461.7^{*} (25.5) | 398.0 (108.4) | 563.7 (53.9) | 812.2^{##} (23.7) | 138.6 ⁵ (3.3) | 803.9 (0.6) |
| IL-8 | 255.3 (99.6) | 1457 (111.7) | 1045 ^{*} (64.2) | 664.6 ^{**} (107.8) | 1211 (56.7) | 1406 ^{#} (36.1) | 371.7 ⁵⁵ (5.8) | 1595 (13.3) |
| TNF | 628.2 (182.6) | 7929 (108.0) | 5558 ^{***} (243.1) | 4044 ^{***} (350.6) | 6603 ^{**} (235.6) | 9600^{##} (128.4) | 3241 ⁵⁵ (280.6) | 7186 (174.8) |
| G-CSF | 730.0 (21.1) | 5230 (109.5) | 1304 ^{***} (296.2) | 2211 ^{***} (24.9) | 1047 ^{***} (121.9) | 3882^{##} (82.2) | 1032 ⁵⁵ (21.4) | 1214 (29.9) |

Statistical comparisons: *, P<0.05; **, P<0.01; ***, P<0.001 compared with LPS/IFN-γ alone ; ##, P<0.001 compared with LPS/IFN-γ + CR845 at 50 nM §, P<0.05 compared with LPS/IFN-γ + CR845 at 50 nM; ¶, P<0.005 compared with LPS/IFN-γ; ¶¶, P<0.001 compared with LPS/IFN-γ

A second human in vitro model of inflammation using synoviocytes is based on the knowledge that TNF-alpha is a pro-inflammatory cytokine and a major target of existing biologic agents for the treatment of rheumatoid arthritis. Accordingly, KTAC compounds of the invention can be assessed for anti-inflammatory activity in synoviocytes cultured from surgically ablated synovial tissue from rheumatoid arthritis patients. Using standard tissue culture methods, human rheumatoid arthritis (RA) synoviocytes obtained from tissue donors (N=3 donors per condition) were stimulated with a combination of interferon-gamma and monoclonal antibody to human CD40 in the presence or absence of CR845 to induce release of the cytokine TNF-alpha over a 48 hour period. Concentrations of TNF-alpha are then measured in tissue culture supernatants by multiplex immunoassay. Standardizing the CD-40/interfereron-gamma stimulated release of TNF-alpha to a value of 100, and using the immunosuppressive corticosteroid budesonide as a positive control, TNF-alpha release was suppressed to a value of -25 with budesonide, with dose-dependent suppression to values of 20 and -25 by concentrations of 0.1 and 0.3 nM CR845, respectively, compared to vehicle (p<0.001 one-way ANOVA), confirming the anti-inflammatory activity, in clinically relevant human disease tissue cells, of a Ka released by conjugates provided by the invention.

### In Vivo Anti-Inflammatory Activity of KTAC Components

The anti-inflammatory activity of compounds of the invention can be further assessed with well-established and validated in vivo rodent models of inflammatory disease.

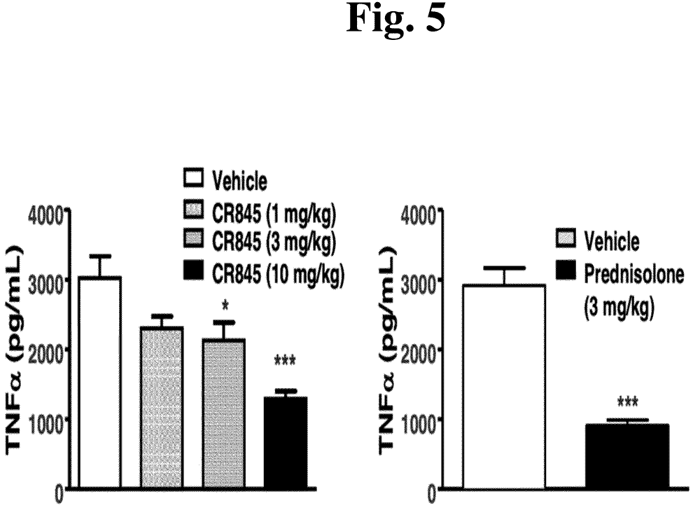

In a mouse model, the Kₐ CR845, vehicle, or the active control prednisolone (3 mg/kg) are administered subcutaneously (SC) in female Balb/c mice. Thirty minutes later, mice were injected IP with the bacterial lipopolysaccharide (LPS, 1 mg/kg) and serum samples collected 2 hours post-LPS treatment (n = 8/group). TNF-alpha levels in serum were determined by ELISA. Doses of 3 and 10 mg/kg produced a dose-dependent suppression of TNF-alpha that is statistically significant (* and *** p<0.05 and 0.001 vs. vehicle; one-way ANOVA followed by Dunnett test), providing a baseline value for comparison to conjugate compounds of the invention (FIG. 5).

To assess the activity of the Ka CR845 against a broader range of inflammatory cytokines, female Balb/c mice were given either CR845 (10 mg/kg, SC) or prednisolone (3 mg/kg, IP) 30 min prior to LPS challenge (1 mg/kg, IP) and sacrificed 2 hr post-LPS challenge to analyze serum samples via Luminex for levels of various cytokines. Data were summarized as percent reduction from vehicle-treated controls (n = 8/group). (*p<0.05 vs. vehicle; unpaired t-test). CR845 SC significantly reduces the release of multiple pro-inflammatory cytokines (TNF, IL-1β, IL-2, IL-12, and MIP-1β) induced by administration of (LPS), with a level of reduction comparable to the clinically used gold standard anti-inflammatory agent, prednisolone (Table 2;*p<0.05 vs. vehicle; unpaired t-test).

TABLE 2

Reduction of LPS-Induced Cytokine Release in Mice Pretreated with SC CR845

| Cytokine | Prednisolone (3 mg/kg, SC) % Inhibition | CR845 (10 mg/kg, SC) % Inhibition |
|---|---|---|
| TNFα | 64* | 47* |
| IL-1β | 39* | 26* |
| IL-2 | 24* | 23* |
| MIP-1β | 19* | 19* |
| IL-12 (p40/p70) | 45* | 23* |
| MIP-1α | 35* | 19 |
| IL-1α | 50* | -37 |
| IL-4 | 41* | 21 |
| IL-10 | 59* | 26 |
| IL-6 | -2 | -10 |
| MCP-1 | -14 | -11 |
| GM-CSF | 10 | 7 |

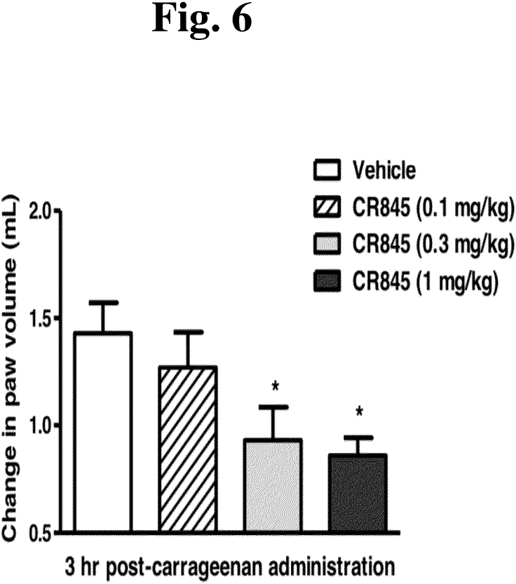

In a rat model of inflammatory disease, intraplantar injection of carrageenan in one hind paw is commonly used to produce acute inflammation, resulting in the swelling of the inoculated paw (Stein, Millan et al. 1988). Animals were administered CR845 IV (tail vein, 1 mL/kg) 30 min prior to intraplantar carrageenan administration (100 µL 2% carrageenan into left hind paw; n=6/group). Paw volumes were assessed 3.5 hr post treatment (i.e., 3 hr post-carrageenan injection). Data were expressed as change in paw volume (inflamed - non-inflamed) as determined by volume displacement with a plethysmometer, and compared to the effects of ibuprofen and prednisolone given SC and IP, respectively. Prednisolone data are from the same laboratory and obtained under similar conditions, but are included as a historical reference since they were obtained prior to data for CR845 and ibuprofen (N = 6-8 male SD rats per group). CR845 IV significantly reduces carrageenan-induced paw swelling, with a minimum effective IV dose of 0.3 mg/kg at 3.5 hr post-injection (FIG. 6; * denotes p < 0.05 vs. vehicle, one-way ANOVA followed by Dunnett test). These data indicate that a Ka of the invention, CR845, has potent anti-inflammatory activity in a second, mechanistically different rodent model of inflammation, and provides an additional baseline against which to assess the anti-inflammatory activity of compounds of the invention.

The disclosures of each of the U.S. patents and the literature references cited in this specification are incorporated by reference herein in their entireties. In the event that any definition or description contained found in one or more of these references is in conflict with the corresponding definition or description herein, then the definition or description disclosed herein is intended.

The examples and embodiments provided herein are for illustration purposes only and are not intended to limit the scope of the invention, the full breadth of which will be readily recognized by those of skill in the art.

1. A conjugate molecule having the structure of formula I: 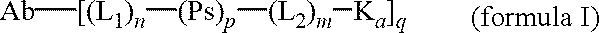
wherein Ab is a therapeutic antibody/therapeutic antibody fragment having a binding site for a tissue specific antigen or a binding site for an antigen overexpressed in a disease or condition;
wherein L₁ and L₂ are linkers; Ps is a linker comprising at least one protease cleavage site and Kₐ comprises a kappa opioid receptor agonist peptide;
wherein n and m are each independently 0 or 1; p is 0 or an integer from 1 to about 10 and q is an integer from 1 to about 10;
wherein when p = 0, (n + m) ≥ 1 and at least one of (L₁)ₙ and (L₂)ₘ comprises an acid labile moiety;
and provided that when n and m are each zero, then p = 1.

2. The conjugate molecule according to claim 1, wherein the kappa opioid receptor agonist peptide, Kₐ comprises one or more D-amino acids.

3. The conjugate molecule according to claim 2, wherein the kappa opioid receptor agonist peptide, Kₐ comprises a D-amino acid tetrapeptide amide.

4. The conjugate molecule according to claim 3, wherein the D-amino acid tetrapeptide amide is CR845.

5. The conjugate molecule according to claim 1, wherein the antibody/antibody fragment, Ab selectively/specifically binds to an antigen specific to an inflammatory tissue.

6. The conjugate molecule according to claim 1, wherein the antibody/antibody fragment selectively/specifically binds to an antigen overexpressed in a disease or condition.

7. The conjugate molecule according to claim 1, wherein the antibody/antibody fragment is a therapeutic antibody/therapeutic antibody fragment.

8. The conjugate molecule according to claim 7, wherein the therapeutic antibody/therapeutic antibody fragment selectively/specifically binds to an antigen overexpressed in a disease or condition.

9. The conjugate molecule according to claim 8, wherein the disease or condition is an inflammatory disease or condition.

10. The conjugate molecule according to claim 9, wherein the inflammatory disease or condition comprises pruritus.

11. The conjugate molecule according to claim 1, wherein at least one protease cleavage site is cleavable by a tissue specific protease.

12. The conjugate molecule according to claim 1, wherein the at least one protease cleavage site comprises a protease cleavage site cleavable by a protease selected from the group consisting of a neutral protease, a serine protease and a matrix metalloprotease.

13. The conjugate molecule according to claim 12, wherein the at least one protease cleavage site comprises a protease cleavage site cleavable by a protease selected from the group consisting of bacillolysin, dispase, mast cell serine protease, chymase, chymotrypsin, trypsin, tryptase, subtilisin, signal peptidase, matrix metalloproteinase 1, matrix metalloproteinase 2, matrix metalloproteinase 3 and matrix metalloproteinase 7.

14. The conjugate molecule according to claim 1, wherein (L₁)ₙ—(Ps)ₚ—(L₂)ₘ comprises an acid labile linkage.

15. A method of treating a disease or condition, the method comprising administering to a patient in need thereof an effective amount of a conjugate molecule according to claim 1.

16. The method according to claim 15, wherein the disease or condition comprises inflammation.

17. The method according to claim 16, wherein the inflammatory disease or condition comprises inflammation and pruritus.

18. The method according to claim 16, wherein the kappa opioid receptor agonist peptide comprises CR845.

19. A pharmaceutical composition comprising the conjugate molecule according to claim 1 and a pharmaceutically acceptable excipient or carrier.

20. The pharmaceutical composition according to claim 16, wherein the kappa opioid receptor agonist peptide comprises CR845.
